(19) United States
(12) Patent Application Publication
(10) Pub. No.: US 2013/0171163 A1
(43) Pub. Date: Jul. 4, 2013

(54) Inhibition of LAR Phosphatase to Enhance Therapeutic Angiogenesis

(71) Applicants: Marschall Runge, Chapel Hill, NC (US); Nageswara Madamanchi, Durham, NC (US)

(72) Inventors: Marschall Runge, Chapel Hill, NC (US); Nageswara Madamanchi, Durham, NC (US)

(21) Appl. No.: 13/813,964

(22) PCT Filed: Aug. 5, 2011
(86) PCT No.: PCT/US11/46719

§ 371 Date: Mar. 6, 2013

Related U.S. Application Data
(60) Provisional application No. 61/371,185, filed on Aug. 6, 2010.

(51) Int. Cl. A61K 39/395; A61K 31/7088; A61K 31/713

(52) U.S. Cl. CPC A61K 39/3955; A61K 31/713; A61K 31/7088
USPC 424/146.1; 514/44 A; 424/158.1; 514/44 R; 435/7.1

(57) ABSTRACT

The present invention relates to the regulation of angiogenesis and arteriogenesis by leukocyte antigen-related protein tyrosine phosphatase (LAR). The invention further relates to the use of inhibitors of LAR expression and/or activity to stimulate angiogenesis and/or arteriogenesis.

### STATEMENT OF PRIORITY

This application claims the benefit of U.S. Provisional Application Ser. No. 61/371,185, filed Aug. 6, 2010, the entire contents of which is incorporated by reference herein.

### STATEMENT OF FEDERAL SUPPORT

This invention was made, in part, with government support under grant numbers HL-057352 and AG-024282 from the National Institutes of Health. The United States government has certain rights to this invention.

### FIELD OF THE INVENTION

The present invention relates to the regulation of angiogenesis and arteriogenesis by leukocyte antigen-related protein tyrosine phosphatase (LAR). The invention further relates to the use of inhibitors of LAR expression and/or activity to stimulate angiogenesis and/or arteriogenesis.

### BACKGROUND OF THE INVENTION

Diabetes now affects nearly 24 million people in the United States (Centers for Disease Control and Prevention. 2007 National Diabetes Fact Sheet. Atlanta, Ga.: U.S. Department of Health and Human Services, Centers for Disease Control and Prevention, 2008) and diabetic patients are at a 10-20 fold increased risk of developing critical limb ischemia (Hochberg et al., Ann. Vasc. Surg. 15:388 (2001)). In fact, diabetes and its sequelae account for over half of all amputations in the United States, with most being lower-extremity amputations. One potential therapeutic approach would be to increase blood flow and decrease ischemia by stimulating angiogenesis in the ischemic region. Studies aimed at enhancing angiogenesis by the delivery of vascular endothelial growth factor (VEGF) have largely been unsuccessful (Uchida et al., Curr. Pharm. Des. 15:411 (2009)). Increased protein tyrosine phosphatase (PTP) activity has been reported in type II diabetes (Zhang, Curr. Opin. Chem. Biol. 5:416 (2001)) and peripheral ischemic diseases (Sugano et al., J. Cardiovasc. Pharmacol. 44:460 (2004)).

Though PTP1B is the major negative regulator of insulin signaling (Elchebly et al., Science 283:1544 (1999)), several studies have shown that leukocyte antigen-related (LAR) PTP also negatively regulates insulin signaling (Ahmad et al., J. Clin. Invest. 95:2806 (1995); Ahmad et al., J. Biol. Chem. 272:448 (1997)). LAR is a receptor type IIA PTP with a broad tissue distribution (Streuli et al., EMBO J. 11:897 (1992)). It is expressed on the cell surface as a complex of non-covalently associated 150-kDa extracellular (E) subunit and an 85-kDa phosphatase (P) subunit, which is generated by the action of an endogenous protease on a 200-kDa proprotein (Streuli et al., EMBO J. 11:897 (1992)). The E-subunit, representing the amino terminus of the protein, is modified by N-linked glycosylation whereas the C-terminal P-subunit contains a short ectodomain, a transmembrane domain, and two tandem phosphatase domains. The membrane-proximal phosphatase domain exhibits enzyme activity in vitro.

It has been recently demonstrated that LAR binds to insulin-like growth-factor-1 receptor (IGF-1R) in vascular smooth muscle cells (VSMCs), and absence of LAR enhances IGF-1-induced IGF-1R phosphorylation (Niu et al., J. Biol. Chem. 282:19808 (2007)). Further, LAR deficiency in mice resulted in a significant decrease in body weight. Several cell culture and mouse studies suggest that IGF-1R is an important regulator of angiogenesis. IGF-1R activation was observed in various types of cancer along with increased tumor angiogenesis and increased expression of VEGF (Reinmuth et al., Clin. Cancer Res. 8:3259 (2002); Stoeltzing et al., Am. J. Pathol. 163:1001 (2003)). Increase in VEGF expression, as a result of constitutive IGF-1R activation, in pancreatic cancer cells is mediated by hypoxia-inducible factor-1α, and suppression of IGF-1R function by transfection with a dominant negative plasmid inhibited angiogenesis and pancreatic tumor growth (Stoeltzing et al., Am. J. Pathol. 163:1001 (2003)). In addition, IGF-1R inhibitors significantly reduced VEGF expression and neovascularization in mouse models of angiogenesis (Moser et al., Eur. J. Cancer 44:1577 (2008); Economou et al., Acta Ophthalmol. 86 Thesis 4:42 (2008)). IGF-1, the principal ligand for IGF-1R, also regulates migration and angiogenesis of endothelial cells (ECs) (Shigematsu et al., Endocr. J. 46 (suppl):S59 (1999)). Deletion of IGF-1R in skeletal muscle of mice markedly impaired glucose tolerance, and these mice developed type II diabetes at six months of age (Su et al., Am. J. Physiol. Heart Circ. Physiol. 284:H1429 (2003)). Several epidemiologic studies indicate that IGF-1 contributes to glucose homeostasis (Clemmons, Curr. Opin. Pharmacol. 6:620 (2006)). Because the relatively high free IGF-1 concentrations required to detect insulin-sensitizing effects are associated with a high incidence of side effects, Phase III clinical trials of IGF-1 in diabetes have not been completed. Instead, strategies to activate IGF-1R, without administering IGF-1, to enhance insulin sensitivity are the subject of ongoing experimental efforts (Clemmons, Nat. Rev. Drug Discov. 6:821 (2007)).

The present invention addresses previous shortcomings in the art by identifying a regulatory role for LAR in angiogenesis and providing methods for regulating angiogenesis and/or arteriogenesis in a subject.

### SUMMARY OF THE INVENTION

The present invention is based, in part, on the identification of the regulatory role played by LAR in angiogenesis and arteriogenesis. The discovery that inhibition of LAR leads to stimulation of angiogenesis and arteriogenesis provides methods for stimulating these processes in a subject in need thereof.

Accordingly, as one aspect, the invention provides a method of increasing the angiogenic potential of a cell (e.g., an endothelial cell), comprising decreasing the expression and/or activity of LAR in the cell.

Another aspect of the invention relates to a method of increasing angiogenesis and/or arteriogenesis in a tissue of a subject, comprising decreasing the expression and/or activity of LAR in said tissue of the subject.

A further aspect of the invention relates to a method of treating or preventing ischemia in a tissue of a subject, comprising decreasing the expression and/or activity of LAR in the tissue of the subject.

An additional aspect of the invention relates to a method of identifying a compound that regulates angiogenesis and/or arteriogenesis, comprising determining the expression and/or activity of LAR in the presence and absence of a test compound, and selecting a compound that increases or decreases the level of expression and/or activity of LAR relative to the level in the absence of the compound, thereby identifying the compound as a compound that regulates angiogenesis.

These and other aspects of the invention are set forth in more detail in the description of the invention below.

### BRIEF DESCRIPTION OF THE DRAWINGS

FIGS. 1A-1E show LAR overexpression inhibits, whereas LAR knockdown enhances, IGF-1-induced autophosphorylation of IGF-1Rβ. A, HUVECs were infected with Adβ-gal or AdLAR and LAR activity (upper panel) was measured using 6,8-difluoro-4-methyl-umbelliferyl phosphate as a substrate. Data presented are mean±SEM (n=3) and representative of three separate experiments (*P<0.05 vs. HUVECs infected with Adβ-gal). LAR protein expression (lower panel) was analyzed by Western blotting with anti-LAR monoclonal antibody (LAR mAb) or β-actin antibody (loading control). B, HUVECs infected with various MOI of Adβ-gal or AdLAR were quiesced overnight and treated without or with 100 ng/mL IGF-1 for 10 min, and cell lysates were immunoprecipitated with anti-IGF-1Rβ antibody, and Western analysis was performed with anti-phosphotyrosine (PY20), anti-IGF-1Rβ, anti-LAR mAb or anti-β-actin antibody. C, Densitometric analysis of IGF-1Rβ autophosphorylation (mean±SEM; n=3; *P<0.05, **P<0.01 vs. AdLAR infected HUVECs treated with IGF-1). D, Control HUVECs and HUVECs transfected with either scrambled siRNA or LAR siRNA were quiesced overnight and left either untreated or treated with 100 ng/mL IGF-1 for 10 min. HUVEC lysates were immunoprecipitated with anti-IGF-1Rβ antibody, and Western analysis was performed with anti-phosphotyrosine (PY20), anti-IGF-1Rβ, anti-LAR mAb or anti-β-actin antibody. E, Densitometric analysis of IGF-1Rβ autophosphorylation (mean±SEM; n=3; *P<0.01 vs. control or scrambled siRNA transfected HUVECs treated with IGF-1).

FIGS. 2A-2B show LAR overexpression does not affect VEGF-induced phosphorylation of VEGFR2 and downstream signaling moieties. A, HUVECs infected with Adβ-gal or AdLAR were treated without or with 20 ng/mL VEGF for 10 min, and cell lysates were analyzed by Western analysis with anti-phospho VEGFR2 (pVEGFR2) antibody. The same membrane was reprobed with anti-phospho Akt (pAkt), anti-Akt, anti-phospho eNOS (p-eNOS), anti-eNOS, anti-phospho ERK1/2 (pERK1/2), anti-ERK1/2, anti-LAR mAb or anti-β-actin antibody. B, Densitometric analysis of pVEGFR2, pAKT, p-eNOS and pERK1/2 levels normalized to their respective nonphosphorylated proteins (mean±SEM; n=3).

FIGS. 3A-3B show knockdown of LAR with siRNA significantly decreases LAR protein expression in HUVECs. A, HUVECs were transfected with scrambled siRNA or LAR siRNA and cell lysates were analyzed by Western blotting with anti-LAR mAb or anti-β-actin antibody. B, Densitometric analysis of LAR protein levels normalized to β-actin protein levels. Data represent as fold change from the scrambled siRNA control (mean±SEM; n=3; *P<0.05 vs. scrambled RNA transfection).

FIGS. 4A-4B show LAR knockdown does not affect VEGF-induced activation of VEGFR2 downstream signaling moieties. A, Control HUVECs and HUVECs transfected with either scrambled siRNA or LAR siRNA were quiesced overnight and either left untreated or treated with 20 ng/mL VEGF for 10 min. HUVEC lysates were analyzed by Western blotting with anti-pAkt, anti-Akt, anti-p-eNOS, anti-eNOS, anti-pERK1/2, anti-ERK1/2, anti-LAR mAb or anti-β-actin antibody. B, Densitometric analysis of pAKT, p-eNOS and pERK1/2 levels normalized to their respective nonphosphorylated proteins (mean±SEM; n=3).

FIGS. 5A-5F show LAR associates with IGF-1Rβ, but not with VEGFR2 or VEGFR3. A, Lysates from HUVECs stimulated with or without IGF-1 (100 ng/ml) for 10 min were immunoprecipitated with polyclonal antibodies against cytoplasmic LAR (anti-cLAR) or control goat IgG, followed by immunoblotting with anti-IGF-1Rβ (top) or anti-cLAR antibody (bottom). B, Densitometric analysis of LAR-associated IGF-1Rβ levels without or with IGF-1 treatment (mean±SEM; n=3; *P<0.05 vs. control). C, Lysates from HUVECs stimulated with or without VEGF (20 ng/ml) for 10 min were immunoprecipitated with polyclonal antibodies against anti-cLAR or control goat IgG, followed by immunoblotting with anti-VEGFR2 (top), VEGFR3 (middle) or anti-cLAR antibody (bottom). D, Equal amounts of GST or GST-cLAR fusion protein were immobilized on glutathione-Sepharose beads and then incubated with HUVEC lysates containing equal amount of protein. GST or GST-cLAR-associated proteins were analyzed by Western blotting using anti-IGF-1Rβ antibody. The same membrane was reprobed with anti-GST antibody (E), anti-VEGFR2 (F, top) or anti-VEGFR3 (F, bottom) antibodies.

FIG. 6 shows that LAR co-localizes with phosphorylated IGF-1Rβ in HUVEC. HUVECs were treated with IGF-1 (100 ng/ml) for 10 min and then fixed. Immunostaining for phospho-IGF-1Rβ (red) and LAR (green) was carried out using antibodies against phospho-IGF-1Rβ (pYpY1135/1136) and cytoplasmic LAR. Yellow staining (arrows) indicates co-localization of LAR with phosphorylated IGF-1Rβ. Nuclei are counterstained with DAPI (blue) (scale bar: 10 μm).

FIGS. 7A-7D show LAR overexpression inhibits, whereas LAR knockdown enhances, IGF-1Rβ downstream signals. A, HUVEC expressing Adβ-gal or AdLAR were treated without or with 100 ng/mL IGF-1 for the indicated times and cell lysates were analyzed by Western blotting with anti-phospho Akt (pAkt; Ser 473), anti-Akt, anti-phospho eNOS (p-eNOS; (Ser1177), anti-eNOS, anti-phospho ERK1/2 (pERK1/2; Thr202/Tyr204), anti-ERK1/2, anti-LAR mAb or anti-β-actin antibody. B, Densitometric analysis of pAkt, p-eNOS and pERK1/2 levels normalized to their respective nonphosphorylated proteins (mean±SEM; n=3; *P<0.001 vs AdLAR infected HUVECs without IGF-1 treatment). C, Control and HUVECs expressing scrambled siRNA or LAR siRNA were quiesced overnight and either left untreated or treated with 100 ng/mL IGF-1 for 10 min and cell lysates were analyzed by Western blotting with anti-pAkt, anti-Akt, anti-p-eNOS, anti-eNOS, anti-pERK1/2, anti-ERK1/2, anti-LAR mAb or anti-β-actin antibody. D, Densitometric analysis of pAkt, p-eNOS and pERK1/2 levels normalized to their respective nonphosphorylated proteins (mean±SEM; n=3; *P<0.01 vs control or scrambled siRNA transfected HUVECs treated with IGF-1).

FIGS. 8A-8E show IGF-1, but not VEGF, induced proliferation and migration are inhibited in HUVECs overexpressing LAR. Control HUVECs and HUVECs infected with AdLAR or Adβ-gal, were quiesced and then treated with 100 ng/mL IGF-1 (A) or 20 ng/mL VEGF (B) for 18 h. [³H]-thymidine incorporation was measured during the last 3 h of treatment. Data presented are mean±SEM (n=3) and representative of three separate experiments (*P<0.05 vs control and Adβ-gal infected cells). Cell migration assay was performed with control, AdLAR and Adβ-gal infected HUVECs with or without IGF-1 or VEGF treatment. The extent of cell migration into the wounded area was photographed under phase-contrast microscopy at 0 and after 16 h (C). Quantitative analysis of cell migration in HUVECs treated with IGF-1 (D) or VEGF (E). The mean distance migrated by HUVECs is quantified (average of 5 independent microscope fields for each of 3 independent experiments; mean±SEM; *P<0.05 vs Adβ-gal-infected or control HUVECs treated with IGF-1; D).

FIGS. 9A-9D show LAR overexpression inhibits, whereas LAR knockdown enhances IGF-1 but not VEGF induced endothelial tube formation in vitro. A, Control HUVECs and HUVECs infected with Adβ-gal or AdLAR were either left untreated or treated with 100 ng/mL IGF-1 or 20 ng/mL VEGF. The HUVECs were then overlaid on growth factor-reduced Matrigel and incubated for 5 h. Entire microscopic fields of view of representative experiments are shown. B, Quantification of tube length. Total tube length was measured using NIH Image J. Measurements were taken from 5-7 microscopic fields per treatment and the average of three replicates (mean±SEM; n=3; *P<0.001 vs control or Adβ-gal infected HUVECs treated with IGF-1). Data are representative of three separate experiments. C, Control HUVECs and HUVECs transfected with either scrambled siRNA or LAR siRNA were either left untreated or treated with 100 ng/mL IGF-1 or 20 ng/mL VEGF. The HUVECs were then overlaid on growth factor-reduced Matrigel and incubated for 5 h. Entire microscopic fields of view of representative experiments are shown. D, Measurement of total tube length and recording of the data were as described above (mean±SEM; n=3; *P<0.001 vs control or scrambled siRNA transfected HUVECs treated with IGF-1).

FIGS. 10A-10C show that suppression of LAR expression with LAR siRNA enhances IGF-1 induced proliferation and migration of HUVEC. A, Control HUVECs and HUVECs transfected with scrambled siRNA or LAR siRNA were quiesced and then treated without or with 100 ng/mL IGF-1 for 18 h. [³H]-thymidine incorporation was measured during the last 3 h of treatment. Data presented are mean±SEM (n=3) and representative of three separate experiments (*P<0.01 vs. respective controls; **P<0.001 vs. respective control; ***P<0.01 vs. scrambled siRNA transfected HUVECs treated with IGF-1). B, The extent of cell migration into the wounded area was photographed under phase-contrast microscopy after 16 h. C, The mean distance migrated by HUVECs was quantified (average of 5 independent microscope fields for each of three independent experiments; mean±SEM; *P<0.05 vs. scrambled siRNA transfected or control HUVECs treated with IGF-1).

FIGS. 11A-11D show SH-6, an Akt inhibitor, abrogates LAR siRNA stimulated, IGF-1-induced activation of Akt, eNOS, and endothelial tube formation, but not ERK1/2. A, HUVECs expressing scrambled siRNA or LAR siRNA were quiesced overnight, pretreated with 1 μmol/L SH-6 and then either left untreated or treated with 100 ng/mL IGF-1 for 10 min and cell lysates were analyzed by Western blotting with anti-pAkt, anti-Akt, anti-p-eNOS, anti-eNOS, anti-pERK1/2, anti-ERK1/2, anti-LAR mAb or anti-β-actin antibody. B, Densitometric analysis of pAkt, p-eNOS and pERK1/2 levels normalized to their respective nonphosphorylated proteins (mean±SEM; n=3; *P<0.05 vs. scrambled siRNA infected HUVECs treated with IGF-1). C, Control HUVECs and HUVECs transfected with either scrambled siRNA or LAR siRNA were quiesced, pretreated with or without SH-6 for 30 min and then were either left untreated or treated with 100 ng/mL IGF-1 or 20 ng/mL VEGF. The HUVECs were then overlaid on growth factor-reduced Matrigel and incubated for 5 h. Entire microscopic fields of view of representative experiments are shown. D, Total tube length was measured using NIH Image J. Measurements were taken from 5-7 microscopic fields per treatment and the average of three replicates (mean±SEM; n=3; *P<0.05 compared with control or scrambled siRNA infected HUVECs treated with IGF-1). Data are representative of three separate experiments.

FIGS. 12A-12E show LAR^{-/-} mice show enhanced angiogenesis ex vivo and in vivo. A, Representative photomicrographs of aortic ring cultures from both wild-type and LAR^{-/-} mice embedded in growth factor-reduced Matrigel and incubated with or without IGF-1 for 10 days. B, Lengths of capillaries were quantitated with NIH Image J. Data are represented as mean±SEM (n=6). *P<0.05 vs. untreated wild-type aortic rings; **P<0.01 vs untreated LAR^{-/-} aortic rings; ***P<0.001 vs. IGF-1-treated wild-type aortic rings; C, Photographs of excised representative Matrigel plugs placed in wild-type or LAR^{-/-} mice that were either untreated (FGF+heparin) or treated with IGF-1 (IGF-1+FGF+heparin). D, Hemoglobin content/gm Matrigel (mean±SEM; n=6; *P<0.05 vs. wild-type mice treated with IGF-1). E, Massons trichrome-stained sections of Matrigel plugs (×20).

FIGS. 13A-13F show that PPP inhibits IGF-1-induced phosphorylation in HUVECs, enhanced IGF-1-induced endothelial cord formation in HUVECS transfected with LAR siRNA and enhanced ex vivo angiogenesis in LAR^{-/-} mice. A, HUVECs were quiesced overnight, pretreated with PPP or DMSO for 1 h and then treated without or with 100 ng/mL IGF-1 for 10 min. Cell lysates were immunoprecipitated (IP) with anti-IGF-1Rβ antibody, and Western analysis was performed with anti-phosphotyrosine (pY20) antibody (upper panel). Cell lysates were also analyzed using antibody against phospho-IGF-1Rβ (pYpY1135/1136) (middle panel) and IGF-1Rβ (lower panel). B, Densitometric analysis of IGF-1Rβ autophosphorylation (mean±SEM; n=3; *P<0.05 vs. control; **P<0.01 vs. IGF-1 treated cells). C, HUVECs transfected with scrambled siRNA or LAR siRNA were overlaid on growth factor-reduced Matrigel. Cells were pretreated with PPP (1 μM) or DMSO for 1 h, then treated without or with IGF-1 (100 ng/ml) for 5 h and fixed. Photographs were taken at 20× magnification. Bar: 100 μm. D, Quantification of cord length using NIH Image J. Measurements were taken from 5-7 microscopic fields per treatment and the average of three replicates shown (mean±SEM; n=3; *,**P<0.001 vs. scrambled siRNA transfected HUVECs treated with IGF-1;***P<0.001 vs. LAR siRNA transfected HUVECs treated with IGF-1). E, Representative photomicrographs (20×) of aortic rings from both wild-type and LAR^{-/-} mice embedded in growth factor-reduced Matrigel and incubated with or without IGF-1 (100 ng/ml) for 10 days in the presence of PPP (1 μM)/DMSO. Bar: 100 μm. F, Lengths of sprouts were quantified and data represented are mean±SEM (n=6; *P<0.01 vs. wild-type treated with IGF-1; **P<0.05 vs. wild-type treated with IGF-1; ***P<0.001 vs. LAR^{-/-} treated with IGF-1).

FIGS. 14A-14C show that PPP inhibits enhanced IGF-1-induced angiogenesis in LAR^{-/-} mice. Matrigel plugs placed in wild-type or LAR^{-/-} mice that were either untreated (FGF+heparin) or treated with IGF-1 (IGF-1+FGF+heparin) were harvested after 10 days and hemoglobin content was determined (mean±SEM; n=5-7; *P<0.01 vs. LAR^{-/-} mice treated with IGF-1). B, Masson's trichrome-stained sections of Matrigel plugs. Arrows highlight large vessels in the Matrigel plugs. C, Quantification of number of microvessels in Matrigel plugs (mean±SEM; n=5-7; *P<0.001 vs. wild-type mice treated with IGF-1; **P<0.001 vs. LAR^{-/-} mice treated with IGF-1).

FIGS. 15A-15D show LAR deficiency enhances blood flow recovery after femoral artery ligation, A, Laser Doppler perfusion images of adductor thigh region with region of interest (ROI) indicated by dashed lines. B, Quantitation of adductor perfusion measured over ROI. Data are mean±SEM; n=10; *P=0.0024; Two-way ANOVA followed by Dunn-Bonferroni t test. C, Laser Doppler perfusion images of plantar foot with ROI. D, Quantitation of plantar perfusion measured over ROI. Data are mean±SEM; n=10; *P<0.0001; Two-way ANOVA followed by Dunn-Bonferroni t test.

FIGS. 16A-16F show LAR deficiency increases basal capillary number, muscle collateral lumen diameter after femoral artery ligation and basal pial collateral number. A, Lectin-stained capillaries in gastrocnemius muscle. B, Capillary number/muscle fiber number ratios at baseline and 14 days after ligation. Data are mean±SEM; n=10; *P<0.001 vs LAR mice; **P<0.01 vs. nonligated LAR^{-/-} mice; ***P<0.01 vs. LAR^{+/+} mice 14 days after ligation. C, Postmortem Microfil™ casted arteriograms of the pial collateral circulation. Red asterisks denote collaterals between anterior cerebral artery and middle cerebral artery trees. D, Collaterals interconnecting middle and anterior cerebral artery trees were counted in both hemispheres of 12-16-week-old mice. Data are means±SEM; n=8; *P<0.05 vs LAR^{+/+} mice. E, Cyano-Masson-elastin staining of collaterals in gracilis muscle. F, Collateral lumen diameter at baseline and 14 days after ligation. Data are means±SEM; n=10; *P<0.001 vs. nonligated LAR^{+/+} mice; **P<0.001 vs. nonligated LAR^{-/-} mice; ***P<0.001 vs. LAR^{+/+} mice 14 days after ligation.

FIGS. 17A-17F show that LAR PTP expression and activity are increased following hindlimb ischemia in wild-type mice. Hindlimb ischemia was induced by right femoral artery ligation. A, Tissue lysates from skeletal muscles of non-ligated and ligated hindlimbs of wild-type and LAR^{-/-} mice at 7 days after ligation were immunoblotted with anti-LAR, anti-PTP1B, anti-SHP2 and anti-tubulin antibodies; representative data are shown. Each lane indicates a different mouse. B, LAR, PTP1B and SHP-2 protein levels were measured by densitometry and normalized to tubulin. Results are mean±SEM (n=6), *P<0.001 vs. non-ligated control. C, Immunofluorescence detection of LAR (red) around pre-existing and remodeled collaterals in the gracilis muscle (upper) and capillaries in gastrocnemius (lower) day 21 after ligation. Insets show higher magnification of capillaries. D, LAR specific activity in non-ischemic and ischemic tissue of wild-type mice at day 7 after ligation. Results shown are mean±SEM (n=6), *P<0.001 vs. non-ligated control. E, Plasma VEGF levels (n=6; P=0.0578, paired t test). F, Skeletal muscle VEGF levels (mean±SEM; n=6).

FIGS. 18A-18D show that LAR is expressed in endothelial, smooth muscle cells, macrophages and other hematopoietic cells in wild-type mice after femoral artery ligation. Immunofluorescence of paraffin-embedded sections shows partial overlap of LAR (red) and CD31 (green) in collaterals (A) and capillaries (B) in hindlimb day 21 after femoral ligation. Immunofluorescence of hindlimb muscle for LAR (red) and α-SM actin (green) in collaterals demonstrates colocalization (yellow, C). Immunofluorescence 3 days after ligation shows colocalization (yellow) of CD45 (green) and LAR (red) around collaterals (D). Panels A, C, and D represent a section of adductor collateral, and panel B a section of gastrocnemius. Images are shown at 40× magnifications. Scale bar: 50 μm. Tissues were stained with DAPI to identify nuclei (blue).

FIGS. 19A-19G show that suppression of LAR expression using lentiviral LAR shRNA enhances blood flow recovery after femoral artery ligation in db/db mice. A, Laser Doppler perfusion images of plantar region with region of interest (ROI indicated by dashed lines). B, Quantitation of plantar perfusion measured over ROI. Data are mean±SEM; n=5; *P<0.0001; Two-way ANOVA followed by Dunn-Bonferroni t test. C, Immunofluorescence of paraffin embedded sections shows suppression of LAR expression (green) in collaterals and capillaries of gastrocnemius and gracilis, respectively, in hindlimbs of db/db mice 21 days after femoral artery ligation and lentiviral LAR shRNA transduction. D, Collateral remodeling was enhanced in db/db mice transduced with lentiviral LAR shRNA. The paraffin-embedded gracilis muscle sections were stained with cyano-Masson-elastin stain. E, CD31-stained capillaries in gastrocnemius. F, Collateral lumen diameter at baseline and 21 days after ligation. Data are mean±SEM; n=5; *P<0.05 vs. scrambled shRNA transduced mice. G, Capillary number/muscle fiber number ratios at baseline and 21 days after ligation. Data are mean±SEM; n=5; *P<0.05 vs. scrambled shRNA transduced mice.

FIGS. 20A-20C show that LAR PTP deficiency enhances the mobilization of endothelial progenitor cells (EPCs) in the peripheral blood 3 days after femoral artery ligation. A, Cells were initially gated to exclude dead cells, debris, and red blood cells. Subsequent gates were used to select total CD45⁻ cell population and to exclude hematopoietic cells expressing the CD45 antigen. B, Four-quadrant FACS analysis for detecting CD34⁺Flk⁺ cells in the gated CD45⁻ cell population. Upper panels show patterns representative of wild-type mouse and the lower panels are representative of LAR^{-/-} mouse. EPCs are positive for both Flk-1 and CD34. The cells in the upper-right quadrant are the desired EPC population with an immunotype of CD45⁻CD34⁺Flk-1⁺. C, Data are mean±SEM; n=7, *P<0.05 vs. ligated LAR^{+/+} mice.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. §1.822 and established usage.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

All publications, patent applications, patents, and other references mentioned herein are incorporated by reference herein in their entirety.

### I. DEFINITIONS

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±20%, ±10%, ±5%, ±1%, ±0.5%, or even±0.1% of the specified amount.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” as used herein should not be interpreted as equivalent to “comprising.”

The term “regulate,” “regulates,” or “regulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject.

By the terms “treat,” “treating,” or “treatment of,” it is intended that the severity of the subject's condition is reduced or at least partially improved or modified and that some alleviation, mitigation or decrease in at least one clinical symptom is achieved.

“Prevent” or “preventing” or “prevention” refer to prevention or delay of the onset of the disorder and/or a decrease in the severity of the disorder in a subject relative to the severity that would develop in the absence of the methods of the invention. The prevention can be complete, e.g., the total absence of ischemia in a subject. The prevention can also be partial, such that the occurrence of ischemia in a subject is less than that which would have occurred without the present invention.

The term “increasing the angiogenic potential of a cell,” as used herein, refers to an increase in the ability of a cell to participate in angiogenesis and/or arteriogenesis. Angiogenic potential can be measured by any method known in the art or as disclosed herein.

The term “LAR activity,” as used herein, refers to the ability of LAR to remove phosphate groups from a substrate. Phosphatase activity can be measured by any method known in the art or as disclosed herein.

The term “disorder associated with angiogenesis and/or arteriogenesis,” as used herein, refers to any disease, disorder, or condition that can be treated by stimulating angiogenesis and/or arteriogenesis.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention.

An “isolated polynucleotide” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence. An isolated polynucleotide that includes a gene is not a fragment of a chromosome that includes such gene, but rather includes the coding region and regulatory regions associated with the gene, but no additional genes naturally found on the chromosome.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

An isolated cell refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

The term “fragment,” as applied to a polynucleotide, will be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences (i.e., transgenes), e.g., two, three, four, five or more heterologous nucleotide sequences.

Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, and adenovirus vectors. Non-viral vectors include plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

Vectors may be introduced into the desired cells by methods known in the art, e.g., transfection, electroporation, microinjection, transduction, cell fusion, DEAE dextran, calcium phosphate precipitation, lipofection (lysosome fusion), use of a gene gun, or a nucleic acid vector transporter (see, e.g., Wu et al., J. Biol. Chem. 267:963 (1992); Wu et al., J. Biol. Chem. 263:14621 (1988); and Hartmut et al., Canadian Patent Application No. 2,012,311, filed Mar. 15, 1990).

In some embodiments, a polynucleotide of this invention can be delivered to a cell in vivo by lipofection. Synthetic cationic lipids designed to limit the difficulties and dangers encountered with liposome-mediated transfection can be used to prepare liposomes for in vivo transfection of a nucleotide sequence of this invention (Feigner et al., Proc. Natl. Acad. Sci. USA 84:7413 (1987); Mackey, et al., Proc. Natl. Acad. Sci. U.S.A. 85:8027 (1988); and Ulmer et al., Science 259:1745 (1993)). The use of cationic lipids may promote encapsulation of negatively charged nucleic acids, and also promote fusion with negatively charged cell membranes (Feigner et al., Science 337:387 (1989)). Particularly useful lipid compounds and compositions for transfer of nucleic acids are described in International Patent Publications WO95/18863 and WO96/17823, and in U.S. Pat. No. 5,459,127. The use of lipofection to introduce exogenous nucleotide sequences into specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting (Mackey, et al., 1988, supra). Targeted peptides, e.g., hormones or neurotransmitters, and proteins such as antibodies, or non-peptide molecules can be coupled to liposomes chemically.

In various embodiments, other molecules can be used for facilitating delivery of a nucleic acid in vivo, such as a cationic oligopeptide (e.g., WO95/21931), peptides derived from nucleic acid binding proteins (e.g., WO96/25508), and/or a cationic polymer (e.g., WO95/21931).

It is also possible to introduce a vector in vivo as naked nucleic acid (see U.S. Pat. Nos. 5,693,622, 5,589,466 and 5,580,859). Receptor-mediated nucleic acid delivery approaches can also be used (Curiel et al., Hum. Gene Ther. 3:147 (1992); Wu et al., J. Biol. Chem. 262:4429 (1987)).

The term “transfection” or “transduction” means the uptake of exogenous or heterologous nucleic acid (RNA and/or DNA) by a cell. A cell has been “transfected” or “transduced” with an exogenous or heterologous nucleic acid when such nucleic acid has been introduced or delivered inside the cell. A cell has been “transformed” by exogenous or heterologous nucleic acid when the transfected or transduced nucleic acid imparts a phenotypic change in the cell and/or a change in an activity or function of the cell. The transforming nucleic acid can be integrated (covalently linked) into chromosomal DNA making up the genome of the cell or it can be present as a stable plasmid.

As used herein, the terms “protein” and “polypeptide” are used interchangeably and encompass both peptides and proteins, unless indicated otherwise.

A “fusion protein” is a polypeptide produced when two heterologous nucleotide sequences or fragments thereof coding for two (or more) different polypeptides not found fused together in nature are fused together in the correct translational reading frame. Illustrative fusion polypeptides include fusions of a polypeptide of the invention (or a fragment thereof) to all or a portion of glutathione-S-transferase, maltose-binding protein, or a reporter protein (e.g., Green Fluorescent Protein, β-glucuronidase, β-galactosidase, luciferase, etc.), hemagglutinin, c-myc, FLAG epitope, etc.

As used herein, a “functional” polypeptide or “functional fragment” is one that substantially retains at least one biological activity normally associated with that polypeptide (e.g., angiogenic activity, protein binding, ligand or receptor binding). In particular embodiments, the “functional” polypeptide or “functional fragment” substantially retains all of the activities possessed by the unmodified peptide. By “substantially retains” biological activity, it is meant that the polypeptide retains at least about 20%, 30%, 40%, 50%, 60%, 75%, 85%, 90%, 95%, 97%, 98%, 99%, or more, of the biological activity of the native polypeptide (and can even have a higher level of activity than, the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as protein binding and angiogenic activity can be measured using assays that are well known in the art and as described herein.

By the term “express” or “expression” of a polynucleotide coding sequence, it is meant that the sequence is transcribed, and optionally, translated. Typically, according to the present invention, expression of a coding sequence of the invention will result in production of the polypeptide of the invention. The entire expressed polypeptide or fragment can also function in intact cells without purification.

### II. STIMULATION OF ANGIOGENESIS

As one aspect, the invention provides a method of increasing the angiogenic potential of a cell, comprising decreasing the expression and/or activity of LAR in the cell. The cell can be any cell involved in the processes of angiogenesis and arteriogenesis, including endothelial cells, intimal cells, smooth muscle cells, pericytes, fibroblasts, connective tissue cells, etc. The cells may be from established cell lines or primary cells from a subject, e.g., a research animal or a patient. The method can be carried out on cells in vitro, ex vivo, or in vivo.

In particular embodiments, the cell in which LAR expression and/or activity is inhibited is an untransformed endothelial cell or a cell from a endothelial cell line. Endothelial cells and cell lines include, without limitation, HUVEC, HCEC, HGEC, HMEC-1, HUV-ST, ECY304, ECV304, and EA.hy926.

In a further aspect, the invention provides a method of increasing angiogenesis and/or arteriogenesis in a tissue of a subject, comprising decreasing the expression and/or activity of LAR in said tissue of the subject.

In another aspect, the invention provides a method of treating or preventing ischemia in a tissue of a subject, comprising decreasing the expression and/or activity of LAR in the tissue of the subject.

In certain embodiments, the subject is one in need of increased angiogenesis and/or arteriogenesis. For example, the subject may be one that has experienced ischemia, is currently experiencing ischemia, or has a disorder or condition that is likely to lead to an ischemic event such as an infarct or to chronic ischemia in a tissue. For example, the ischemia may be in a part of the body that is more susceptible to ischemia in diseases like diabetes, such as a limb, e.g., a lower limb, e.g., the foot and/or toes. In certain embodiments, the subject is at risk for ischemia, e.g., a subject having a disease that predisposes the subject to ischemia. In one embodiment, the subject has diabetes (e.g., type I or type II) or a pre-diabetic condition. The subject may be suffering from or at risk for one or more complications due to diabetes, such as nephropathy, retinopathy, coronary artery disease, peripheral vascular disease and associated ulcers, gangrene, and/or pain, and/or autonomic dysfunction. In other embodiments, the subject does not have diabetes (e.g., type I or type II). In other embodiments, the subject has cardiovascular or cerebrovascular disease or has experienced ischemia or stroke. In other embodiments, the subject has a graft (e.g., a skin graft) or other transplanted tissue, an anastomosis, a wound, an ulcer, a burn, male pattern baldness, atherosclerosis, ischemic heart tissue, ischemic peripheral tissue (e.g., limb or mesentery ischemia), myocardial or cerebral infarction, or vascular occlusion or stenosis.

In one embodiment of the invention, decreasing the expression and/or activity of LAR comprises decreasing the level of a nucleic acid (DNA or RNA) encoding the polypeptide or the level of expression of the polypeptide from the nucleic acid. Numerous methods for reducing the level and/or expression of polynucleotides in vitro or in vivo are known. For example, the coding and noncoding nucleotide sequences for LAR are known to those of skill in the art and are readily available in sequence databases such as GenBank. Available sequences include, without limitation, human LAR variant 1 (accession number NM_{—}002840), human LAR variant 2 (accession number NM 130440); rat LAR (accession number NM_{—}019249); and mouse LAR (accession number NM_{—}011213), each of which is incorporated by reference in its entirety.

An antisense nucleotide sequence or nucleic acid encoding an antisense nucleotide sequence can be generated to any portion the LAR sequence (including coding and non-coding regions) in accordance with known techniques. The term “antisense nucleotide sequence” or “antisense oligonucleotide” as used herein, refers to a nucleotide sequence that is complementary to a specified DNA or RNA sequence. Antisense oligonucleotides and nucleic acids that express the same can be made in accordance with conventional techniques. See, e.g., U.S. Pat. No. 5,023,243 to Tullis; U.S. Pat. No. 5,149,797 to Pederson et al. The antisense nucleotide sequence can be complementary to the entire nucleotide sequence encoding the polypeptide or a portion thereof of at least 10, 20, 40, 50, 75, 100, 150, 200, 300, or 500 contiguous bases and will reduce the level of polypeptide production.

Those skilled in the art will appreciate that it is not necessary that the antisense nucleotide sequence be fully complementary to the target sequence as long as the degree of sequence similarity is sufficient for the antisense nucleotide sequence to hybridize to its target and reduce production of the polypeptide. As is known in the art, a higher degree of sequence similarity is generally required for short antisense nucleotide sequences, whereas a greater degree of mismatched bases will be tolerated by longer antisense nucleotide sequences.

For example, hybridization of such nucleotide sequences can be carried out under conditions of reduced stringency, medium stringency or even stringent conditions (e.g., conditions represented by a wash stringency of 35-40% formamide with 5×Denhardt's solution, 0.5% SDS and 1×SSPE at 37° C.; conditions represented by a wash stringency of 40-45% formamide with 5×Denhardt's solution, 0.5% SDS, and 1×SSPE at 42° C.; and/or conditions represented by a wash stringency of 50% formamide with 5×Denhardt's solution, 0.5% SDS and 1×SSPE at 42° C., respectively) to the nucleotide sequences specifically disclosed herein. See, e.g., Sambrook et al., Molecular Cloning: A Laboratory Manual 2nd Ed. (Cold Spring Harbor, N.Y., 1989).

In other embodiments, antisense nucleotide sequences of the invention have at least about 70%, 80%, 90%, 95%, 97%, 98% or higher sequence similarity with the complement of the coding sequences specifically disclosed herein and will reduce the level of polypeptide production.

In other embodiments, the antisense nucleotide sequence can be directed against any coding sequence, the silencing of which results in a modulation of LAR.

The length of the antisense nucleotide sequence (i.e., the number of nucleotides therein) is not critical as long as it binds selectively to the intended location and reduces transcription and/or translation of the target sequence, and can be determined in accordance with routine procedures. In general, the antisense nucleotide sequence will be from about eight, ten or twelve nucleotides in length up to about 20, 30, 50, 75 or 100 nucleotides, or longer, in length.

An antisense nucleotide sequence can be constructed using chemical synthesis and enzymatic ligation reactions by procedures known in the art. For example, an antisense nucleotide sequence can be chemically synthesized using naturally occurring nucleotides or various modified nucleotides designed to increase the biological stability of the molecules or to increase the physical stability of the duplex formed between the antisense and sense nucleotide sequences, e.g., phosphorothioate derivatives and acridine substituted nucleotides can be used. Examples of modified nucleotides which can be used to generate the antisense nucleotide sequence include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleotide sequence can be produced using an expression vector into which a nucleic acid has been cloned in an antisense orientation (i.e., RNA transcribed from the inserted nucleic acid will be of an antisense orientation to a target nucleic acid of interest).

The antisense nucleotide sequences of the invention further include nucleotide sequences wherein at least one, or all, of the internucleotide bridging phosphate residues are modified phosphates, such as methyl phosphonates, methyl phosphonothioates, phosphoromorpholidates, phosphoropiperazidates and phosphoramidates. For example, every other one of the internucleotide bridging phosphate residues can be modified as described. In another non-limiting example, the antisense nucleotide sequence is a nucleotide sequence in which one, or all, of the nucleotides contain a 2′ lower alkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides can be modified as described. See also, Furdon et al., Nucleic Acids Res. 17:9193 (1989); Agrawal et al., Proc. Natl. Acad. Sci. USA 87:1401 (1990); Baker et al., Nucleic Acids Res. 18:3537 (1990); Sproat et al., Nucleic Acids Res. 17:3373 (1989); Walder and Walder, Proc. Natl. Acad. Sci. USA 85:5011 (1988); incorporated by reference herein in their entireties for their teaching of methods of making antisense molecules, including those containing modified nucleotide bases).

Triple helix base-pairing methods can also be employed to inhibit production of LAR. Triple helix pairing is believed to work by inhibiting the ability of the double helix to open sufficiently for the binding of polymerases, transcription factors, or regulatory molecules. Recent therapeutic advances using triplex DNA have been described in the literature (e.g., Gee et al., (1994) In: Huber et al., Molecular and Immunologic Approaches, Futura Publishing Co., Mt. Kisco, N.Y.).

RNA interference (RNAi) provides another approach for modulating the expression of LAR. Molecules used for RNAi are short double stranded RNAs that include small interference (si) RNA and small hairpin (sh) RNA, which is cleaved to siRNA. The siRNA can be directed against polynucleotide sequences encoding LAR or any other sequence that results in modulation of the expression of LAR. Examples of siRNA sequences that can be used in the present invention include, without limitation:

(SEQ ID NO: 1)
5′-CCACUAUGCAACGUAACUA-3′;
(SEQ ID NO: 2)
5′-CAGACGAGAUCCUGUUUCA-3′;
and
(SEQ ID NO: 3)
5′-GGCUAAGUCAAGAUCAACA-3′.

RNAi is a mechanism of post-transcriptional gene silencing in which double-stranded RNA (dsRNA) corresponding to a coding sequence of interest is introduced into a cell or an organism, resulting in degradation of the corresponding mRNA. The mechanism by which siRNA achieves gene silencing has been reviewed in Sharp et al., Genes Dev. 15:485 (2001); and Hammond et al., Nature Rev. Gen. 2:110 (2001)). The siRNA effect persists for multiple cell divisions before gene expression is regained. RNAi is therefore a powerful method for making targeted knockouts or “knockdowns” at the RNA level. siRNA has proven successful in human cells, including human embryonic kidney and HeLa cells (see, e.g., Elbashir et al., Nature 411:494 (2001)). In one embodiment, silencing can be induced in mammalian cells by enforcing endogenous expression of RNA hairpins (see Paddison et al., Proc. Natl. Acad. Sci. USA 99:1443 (2002)). In another embodiment, transfection of small (21-23 nt) dsRNA specifically inhibits nucleic acid expression (reviewed in Caplen, Trends Biotechnol. 20:49 (2002)).

siRNA technology utilizes standard molecular biology methods. dsRNA corresponding to all or a part of a target coding sequence to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. Methods of transfection of dsRNA or plasmids engineered to make dsRNA are routine in the art.

MicroRNA (miRNA), single stranded RNA molecules of about 21-23 nucleotides in length, can be used in a similar fashion to siRNA to modulate gene expression (see U.S. Pat. No. 7,217,807).

Silencing effects similar to those produced by siRNA have been reported in mammalian cells with transfection of a mRNA-cDNA hybrid construct (Lin et al., Biochem. Biophys. Res. Commun. 281:639 (2001)), providing yet another strategy for silencing a coding sequence of interest.

The expression of LAR can also be inhibited using ribozymes. Ribozymes are RNA-protein complexes that cleave nucleic acids in a site-specific fashion. Ribozymes have specific catalytic domains that possess endonuclease activity (Kim et al., Proc. Natl. Acad. Sci. USA 84:8788 (1987); Gerlach et al., Nature 328:802 (1987); Forster and Symons, Cell 49:211 (1987)). For example, a large number of ribozymes accelerate phosphoester transfer reactions with a high degree of specificity, often cleaving only one of several phosphoesters in an oligonucleotide substrate (Michel and Westhof, J. Mol. Biol. 216:585 (1990); Reinhold-Hurek and Shub, Nature 357:173 (1992)). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, Nature 338:217 (1989)). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., Proc. Natl. Acad. Sci. USA 88:10591 (1991); Sarver et al., Science 247:1222 (1990); Sioud et al., J. Mol. Biol. 223:831 (1992)).

In another embodiment of the invention, decreasing the expression and/or activity of LAR comprises decreasing the activity of the polypeptide. LAR activity can be modulated by interaction with an antibody or antibody fragment. The antibody or antibody fragment can bind to LAR or to any other polypeptide of interest, as long as the binding between the antibody or the antibody fragment and the target polypeptide results in modulation of the activity of LAR.

The term “antibody” or “antibodies” as used herein refers to all types of immunoglobulins, including IgG, IgM, IgA, IgD, and IgE. The antibody can be monoclonal or polyclonal and can be of any species of origin, including (for example) mouse, rat, rabbit, horse, goat, sheep, camel, or human, or can be a chimeric antibody. See, e.g., Walker et al., Molec. Immunol. 26:403 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980.

Antibody fragments included within the scope of the present invention include, for example, Fab, Fab′, F(ab′)₂, and Fv fragments; domain antibodies, diabodies; vaccibodies, linear antibodies; single-chain antibody molecules; and multispecific antibodies formed from antibody fragments. Such fragments can be produced by known techniques. For example, F(ab′)₂ fragments can be produced by pepsin digestion of the antibody molecule, and Fab fragments can be generated by reducing the disulfide bridges of the F(ab′)₂ fragments. Alternatively, Fab expression libraries can be constructed to allow rapid and easy identification of monoclonal Fab fragments with the desired specificity (Huse et al., Science 254:1275 (1989)).

Antibodies of the invention may be altered or mutated for compatibility with species other than the species in which the antibody was produced. For example, antibodies may be humanized or camelized. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework (FR) regions (i.e., the sequences between the CDR regions) are those of a human immunoglobulin consensus sequence. The humanized antibody optimally also will comprise at least a portion of an immunoglobulin constant region (Fc), typically that of a human immunoglobulin (Jones et al., Nature 321:522 (1986); Riechmann et al., Nature 332:323 (1988); and Presta, Curr. Op. Struct. Biol. 2:593 (1992)).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can essentially be performed following the method of Winter and co-workers (Jones et al., Nature 321:522 (1986); Riechmann et al., Nature 332:323 (1988); Verhoeyen et al., Science 239:1534 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues (e.g., all of the CDRs or a portion thereof) and possibly some FR residues are substituted by residues from analogous sites in rodent antibodies. As used herein, a “portion” of a CDR is defined as one or more of the three loops from each of the light and heavy chain that make up the CDRs (e.g., from 1-6 of the CDRs) or one or more portions of a loop comprising, consisting essentially of, or consisting of at least three contiguous amino acids. For example, the chimeric or humanized antibody may comprise 1, 2, 3, 4, 5, or 6 CDR loops, portions of 1, 2, 3, 4, 5, or 6 CDR loops, or a mixture thereof.

Human antibodies can also be produced using various techniques known in the art, including phage display libraries (Hoogenboom and Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol. 147:86 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779 (1992); Lonberg et al., Nature 368:856 (1994); Morrison, Nature 368:812 (1994); Fishwild et al., Nature Biotechnol. 14:845 (1996); Neuberger, Nature Biotechnol. 14:826 (1996); Lonberg and Huszar, Intern. Rev. Immunol. 13:65 (1995).

Polyclonal antibodies used to carry out the present invention can be produced by immunizing a suitable animal (e.g., rabbit, goat, etc.) with an antigen to which a monoclonal antibody to the target binds, collecting immune serum from the animal, and separating the polyclonal antibodies from the immune serum, in accordance with known procedures.

Monoclonal antibodies used to carry out the present invention can be produced in a hybridoma cell line according to the technique of Kohler and Milstein, Nature 265:495 (1975). For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in E. coli by recombinant techniques known to those skilled in the art. See, e.g., Huse, Science 246:1275 (1989).

Antibodies specific to the target polypeptide can also be obtained by phage display techniques known in the art.

Various immunoassays can be used for screening to identify antibodies having the desired specificity for the polypeptides of this invention. Numerous protocols for competitive binding or immunoradiometric assays using either polyclonal or monoclonal antibodies with established specificity are well known in the art. Such immunoassays typically involve the measurement of complex formation between an antigen and its specific antibody (e.g., antigen/antibody complex formation). A two-site, monoclonal-based immunoassay utilizing monoclonal antibodies reactive to two non-interfering epitopes on the polypeptides or peptides of this invention can be used as well as a competitive binding assay.

Antibodies can be conjugated to a solid support (e.g., beads, plates, slides or wells formed from materials such as latex or polystyrene) in accordance with known techniques. Antibodies can likewise be conjugated to detectable groups such as radiolabels (e.g., ³⁵S, ¹²⁵I, ¹³¹I) enzyme labels (e.g., horseradish peroxidase, alkaline phosphatase), and fluorescence labels (e.g., fluorescein) in accordance with known techniques. Determination of the formation of an antibody/antigen complex in the methods of this invention can be by detection of, for example, precipitation, agglutination, flocculation, radioactivity, color development or change, fluorescence, luminescence, etc., as is well known in the art.

In one embodiment, the activity of LAR is inhibited using aptamers. Recently, small structured single-stranded RNAs, also known as RNA aptamers, have emerged as viable alternatives to small-molecule and antibody-based therapy (Que-Gewirth et al., Gene Ther. 14:283 (2007); Ireson et al., Mol. Cancer. Ther. 5:2957 (2006)). RNA aptamers specifically bind target proteins with high affinity, are quite stable, lack immunogenicity, and elicit biological responses. Aptamers are evolved by means of an iterative selection method called SELEX (systematic evolution of ligands by exponential enrichment) to specifically recognize and tightly bind their targets by means of well-defined complementary three-dimensional structures.

RNA aptamers represent a unique emerging class of therapeutic agents (Que-Gewirth et al., Gene Ther. 14:283 (2007); Ireson et al., Mol. Cancer. Ther. 5:2957 (2006)). They are relatively short (12-30 nucleotide) single-stranded RNA oligonucleotides that assume a stable three-dimensional shape to tightly and specifically bind selected protein targets to elicit a biological response. In contrast to antisense oligonucleotides, RNA aptamers can effectively target extracellular targets. Like antibodies, aptamers possess binding affinities in the low nanomolar to picomolar range. In addition, aptamers are heat stable, lack immunogenicity, and possess minimal interbatch variability. Chemical modifications, such as amino or fluoro substitutions at the 2′ position of pyrimidines, may reduce degradation by nucleases. The biodistribution and clearance of aptamers can also be altered by chemical addition of moieties such as polyethylene glycol and cholesterol. Further, SELEX allows selection from libraries consisting of up to 10¹⁵ ligands to generate high-affinity oligonucleotide ligands to purified biochemical targets.

In another embodiment, the method of decreasing the activity of LAR comprises delivering to a cell or to a subject a compound that decreases the activity of LAR, the compound administered in an amount effective to modulate the activity of LAR. The compound can interact directly with LAR to decrease the activity of the polypeptide. Alternatively, the compound can interact with any other polypeptide, nucleic acid or other molecule if such interaction results in a decrease of the activity of LAR. In certain embodiments, the compound is one disclosed in U.S. Pat. No. 7,115,624, 7,019,026, 6,951,878, 6,410,586, 6,262,044, or 6,225,329, each incorporated herein by reference. In another embodiment, the compound is illudalic acid or analogs thereof such as those disclosed in Ling et al., Acta Pharm. Sinica 45:1385 (2010); each incorporated by reference herein in its entirety. Other compounds include, without limitation, 3S-peptide-I, sodium orthovanadate, sodium pervanadate, RWJ-60475, and LAR wedge domain peptides (Xie et al., J. Biol. Chem. 281:16482 (2006), incorporated by reference herein in its entirety).

The term “compound” as used herein is intended to be interpreted broadly and encompasses organic and inorganic molecules. Organic compounds include, but are not limited to, small molecules, polypeptides, lipids, carbohydrates, coenzymes, aptamers, and nucleic acid molecules (e.g., gene delivery vectors, antisense oligonucleotides, siRNA, all as described above).

Polypeptides include, but are not limited to, antibodies (described in more detail above) and enzymes. Nucleic acids include, but are not limited to, DNA, RNA and DNA-RNA chimeric molecules. Suitable RNA molecules include siRNA, antisense RNA molecules and ribozymes (all of which are described in more detail above). The nucleic acid can further encode any polypeptide such that administration of the nucleic acid and production of the polypeptide results in a decrease of the activity of LAR.

The compound can further be a compound that is identified by any of the screening methods described below.

The compounds of the present invention can optionally be delivered in conjunction with other therapeutic agents. The additional therapeutic agents can be delivered concurrently with the compounds of the invention. As used herein, the word “concurrently” means sufficiently close in time to produce a combined effect (that is, concurrently can be simultaneously, or it can be two or more events occurring within a short time period before or after each other). In one embodiment, the compounds of the invention are administered in conjunction with angiogenesis promoting agents, such as VEGF, platelet-derived growth factor (PDGF), basic FGF (bFGF), epidermal growth factor (EGF), transforming growth factor (TGF), insulin-like growth factors, angiopoietin 1, sphingosine-1-phosphate, and matrix metalloproteinases.

LAR inhibitors that are polypeptides can be delivered to a cell (e.g., an isolated cell or a cell in a subject) in the form of polynucleotides encoding the polypeptides to produce expression of the polypeptide within the cell. Similarly, antisense RNAs, siRNAs, and other inhibitory RNAs can be delivered to a cell (e.g., an isolated cell or a cell in a subject) in the form of polynucleotides encoding the RNA to produce expression of the RNA within the cell. Those skilled in the art will appreciate that the isolated polynucleotides encoding the polypeptides and RNAs of the invention will typically be associated with appropriate expression control sequences, e.g., transcription/translation control signals and polyadenylation signals.

It will further be appreciated that a variety of promoter/enhancer elements can be used depending on the level and tissue-specific expression desired. The promoter can be constitutive or inducible, depending on the pattern of expression desired. The promoter can be native or foreign and can be a natural or a synthetic sequence. By foreign, it is intended that the transcriptional initiation region is not found in the wild-type host into which the transcriptional initiation region is introduced. The promoter is chosen so that it will function in the target cell(s) of interest.

To illustrate, the polypeptide coding sequence can be operatively associated with a cytomegalovirus (CMV) major immediate-early promoter, an albumin promoter, an Elongation Factor 1-α (EF1-α) promoter, a PγK promoter, a MFG promoter, or a Rous sarcoma virus promoter.

Inducible promoter/enhancer elements include hormone-inducible and metal-inducible elements, and other promoters regulated by exogenously supplied compounds, including without limitation, the zinc-inducible metallothionein (MT) promoter; the dexamethasone (Dex)-inducible mouse mammary tumor virus (MMTV) promoter; the T7 polymerase promoter system (see WO 98/10088); the ecdysone insect promoter (No et al., Proc. Natl. Acad. Sci. USA 93:3346 (1996)); the tetracycline-repressible system (Gossen et al., Proc. Natl. Acad. Sci. USA 89:5547 (1992)); the tetracycline-inducible system (Gossen et al., Science 268:1766 (1995); see also Harvey et al., Curr. Opin. Chem. Biol. 2:512 (1998)); the RU486-inducible system (Wang et al., Nat. Biotech. 15:239 (1997); Wang et al., Gene Ther., 4:432 (1997)); and the rapamycin-inducible system (Magari et al., J. Clin. Invest. 100:2865 (1997)).

Other tissue-specific promoters or regulatory promoters include, but are not limited to, promoters that typically confer tissue-specificity in endothelial cells. These include, but are not limited to, promoters for VE-cadherin, PPE-I, PPE-1-3x, TIE-I, TIE-2, Endoglin, von Willebrand, KDR/flk-1, FLT-I, Egr-1, ICAM-1, ICAM-2, VCAM-1, PECAM-I, and aortic carboxypeptidase-like protein (ACLP).

Moreover, specific initiation signals are generally required for efficient translation of inserted polypeptide coding sequences. These translational control sequences, which can include the ATG initiation codon and adjacent sequences, can be of a variety of origins, both natural and synthetic.

The isolated polynucleotide can be incorporated into an expression vector. Expression vectors compatible with various host cells are well known in the art and contain suitable elements for transcription and translation of nucleic acids. Typically, an expression vector contains an “expression cassette,” which includes, in the 5′ to 3′direction, a promoter, a coding sequence encoding the polypeptide operatively associated with the promoter, and, optionally, a termination sequence including a stop signal for RNA polymerase and a polyadenylation signal for polyadenylase.

Non-limiting examples of promoters of this invention include CYC1, HIS3, GAL1, GAL4, GAL10, ADH1, PGK, PHO5, GAPDH, ADC1, TRP1, URA3, LEU2, ENO, TPI, and alkaline phosphatase promoters (useful for expression in Saccharomyces); AOX1 promoter (useful for expression in Pichia); β-lactamase, lac, ara, tet, trp, IP_{L}, IP_{R}, T7, tac, and trc promoters (useful for expression in Escherichia coli); light regulated-, seed specific-, pollen specific-, ovary specific-, pathogenesis or disease related-promoters, cauliflower mosaic virus 35S, CMV 35S minimal, cassaya vein mosaic virus (CsVMV), chlorophyll a/b binding protein, ribulose 1,5-bisphosphate carboxylase, shoot-specific promoters, root specific promoters, chitinase, stress inducible promoters, rice tungro bacilliform virus, plant super-promoter, potato leucine aminopeptidase, nitrate reductase, mannopine synthase, nopaline synthase, ubiquitin, zein protein, and anthocyanin promoters (useful for expression in plant cells).

Further examples of animal and mammalian promoters known in the art include, but are not limited to, the SV40 early (SV40e) promoter region, the promoter contained in the 3′ long terminal repeat (LTR) of Rous sarcoma virus (RSV), the promoters of the E1A or major late promoter (MLP) genes of adenoviruses (Ad), the cytomegalovirus (CMV) early promoter, the herpes simplex virus (HSV) thymidine kinase (TK) promoter, baculovirus IE 1 promoter, elongation factor 1 alpha (EF1) promoter, phosphoglycerate kinase (PGK) promoter, ubiquitin (Ubc) promoter, an albumin promoter, the regulatory sequences of the mouse metallothionein-L promoter and transcriptional control regions, the ubiquitous promoters (HPRT, vimentin, α-actin, tubulin and the like), the promoters of the intermediate filaments (desmin, neurofilaments, keratin, GFAP, and the like), the promoters of therapeutic genes (of the MDR, CFTR or factor VIII type, and the like), pathogenesis and/or disease-related promoters, and promoters that exhibit tissue specificity, such as the elastase I gene control region, which is active in pancreatic acinar cells; the insulin gene control region active in pancreatic beta cells, the immunoglobulin gene control region active in lymphoid cells, the mouse mammary tumor virus control region active in testicular, breast, lymphoid and mast cells; the albumin gene promoter, the Apo AI and Apo AII control regions active in liver, the alpha-fetoprotein gene control region active in liver, the alpha 1-antitrypsin gene control region active in the liver, the beta-globin gene control region active in myeloid cells, the myelin basic protein gene control region active in oligodendrocyte cells in the brain, the myosin light chain-2 gene control region active in skeletal muscle, and the gonadotropic releasing hormone gene control region active in the hypothalamus, the pyruvate kinase promoter, the villin promoter, the promoter of the fatty acid binding intestinal protein, the promoter of smooth muscle cell α-actin, and the like. In addition, any of these expression sequences of this invention can be modified by addition of enhancer and/or regulatory sequences and the like.

Enhancers that may be used in embodiments of the invention include but are not limited to: an SV40 enhancer, a cytomegalovirus (CMV) enhancer, an elongation factor I (EF1) enhancer, yeast enhancers, viral gene enhancers, and the like.

Termination control regions, i.e., terminator or polyadenylation sequences, may be derived from various genes native to the preferred hosts. In some embodiments of the invention, the termination control region may comprise or be derived from a synthetic sequence, a synthetic polyadenylation signal, an SV40 late polyadenylation signal, an SV40 polyadenylation signal, a bovine growth hormone (BGH) polyadenylation signal, viral terminator sequences, or the like.

It will be apparent to those skilled in the art that any suitable vector can be used to deliver the polynucleotide to a cell or subject. The vector can be delivered to cells in vivo. In other embodiments, the vector can be delivered to cells ex vivo, and then cells containing the vector are delivered to the subject. The choice of delivery vector can be made based on a number of factors known in the art, including age and species of the target host, in vitro versus in vivo delivery, level and persistence of expression desired, intended purpose (e.g., for therapy or screening), the target cell or organ, route of delivery, size of the isolated polynucleotide, safety concerns, and the like.

Suitable vectors include plasmid vectors, viral vectors (e.g., retrovirus, alphavirus; vaccinia virus; adenovirus, adeno-associated virus and other parvoviruses, lentivirus, poxvirus, or herpes simplex virus), lipid vectors, poly-lysine vectors, synthetic polyamino polymer vectors, and the like.

Any viral vector that is known in the art can be used in the present invention. Protocols for producing recombinant viral vectors and for using viral vectors for nucleic acid delivery can be found in Ausubel et al., Current Protocols in Molecular Biology (Green Publishing Associates, Inc. and John Wiley & Sons, Inc., New York) and other standard laboratory manuals (e.g., Vectors for Gene Therapy. In: Current Protocols in Human Genetics. John Wiley and Sons, Inc.: 1997).

Non-viral transfer methods can also be employed. Many non-viral methods of nucleic acid transfer rely on normal mechanisms used by mammalian cells for the uptake and intracellular transport of macromolecules. In particular embodiments, non-viral nucleic acid delivery systems rely on endocytic pathways for the uptake of the nucleic acid molecule by the targeted cell. Exemplary nucleic acid delivery systems of this type include liposomal derived systems, poly-lysine conjugates, and artificial viral envelopes.

In particular embodiments, plasmid vectors are used in the practice of the present invention. For example, naked plasmids can be introduced into muscle cells by injection into the tissue. Expression can extend over many months, although the number of positive cells is typically low (Wolff et al., Science 247:247 (1989)). Cationic lipids have been demonstrated to aid in introduction of nucleic acids into some cells in culture (Feigner and Ringold, Nature 337:387 (1989)). Injection of cationic lipid plasmid DNA complexes into the circulation of mice has been shown to result in expression of the DNA in lung (Brigham et al., Am. J. Med. Sci. 298:278 (1989)). One advantage of plasmid DNA is that it can be introduced into non-replicating cells.

In a representative embodiment, a nucleic acid molecule (e.g., a plasmid) can be entrapped in a lipid particle bearing positive charges on its surface and, optionally, tagged with antibodies against cell surface antigens of the target tissue (Mizuno et al., No Shinkei Geka 20:547 (1992); PCT publication WO 91/06309; Japanese patent application 1047381; and European patent publication EP-A-43075).

Liposomes that consist of amphiphilic cationic molecules are useful as non-viral vectors for nucleic acid delivery in vitro and in vivo (reviewed in Crystal, Science 270:404 (1995); Blaese et al., Cancer Gene Ther. 2:291 (1995); Behr et al., Bioconjugate Chem. 5:382 (1994); Remy et al., Bioconjugate Chem. 5:647 (1994); and Gao et al., Gene Therapy 2:710 (1995)). The positively charged liposomes are believed to complex with negatively charged nucleic acids via electrostatic interactions to form lipid:nucleic acid complexes. The lipid:nucleic acid complexes have several advantages as nucleic acid transfer vectors. Unlike viral vectors, the lipid:nucleic acid complexes can be used to transfer expression cassettes of essentially unlimited size. Since the complexes lack proteins, they can evoke fewer immunogenic and inflammatory responses. Moreover, they cannot replicate or recombine to form an infectious agent and have low integration frequency. A number of publications have demonstrated that amphiphilic cationic lipids can mediate nucleic acid delivery in vivo and in vitro (Feigner et al., Proc. Natl. Acad. Sci. USA 84:7413 (1987); Loeffler et al., Meth. Enzymol. 217:599 (1993); Feigner et al., J. Biol. Chem. 269:2550 (1994)).

Several groups have reported the use of amphiphilic cationic lipid:nucleic acid complexes for in vivo transfection both in animals and in humans (reviewed in Gao et al., Gene Therapy 2:710 (1995); Zhu et al., Science 261:209 (1993); and Thierry et al., Proc. Natl. Acad. Sci. USA 92:9742 (1995)). U.S. Pat. No. 6,410,049 describes a method of preparing cationic lipid:nucleic acid complexes that have a prolonged shelf life.

Expression vectors can be designed for expression of polypeptides in prokaryotic or eukaryotic cells. For example, polypeptides can be expressed in bacterial cells such as E. coli, insect cells (e.g., the baculovirus expression system), yeast cells, plant cells or mammalian cells. Some suitable host cells are discussed further in Goeddel, Gene Expression Technology: Methods in Enzymology 185, Academic Press, San Diego, Calif. (1990). Examples of bacterial vectors include pQE70, pQE60, pQE-9 (Qiagen), pBS, pD10, phagescript, psiX174, pbluescript SK, pbsks, pNH8A, pNH16a, pNH18A, pNH46A (Stratagene); ptrc99a, pKK223-3, pKK233-3, pDR540, and pRIT5 (Pharmacia). Examples of vectors for expression in the yeast S. cerevisiae include pYepSec1 (Baldari et al., EMBO J. 6:229 (1987)), pMFa (Kurjan and Herskowitz, Cell 30:933 (1982)), pJRY88 (Schultz et al., Gene 54:113 (1987)), and pYES2 (Invitrogen Corporation, San Diego, Calif.). Baculovirus vectors available for expression of nucleic acids to produce proteins in cultured insect cells (e.g., Sf 9 cells) include the pAc series (Smith et al., Mol. Cell. Biol. 3:2156 (1983)) and the pVL series (Lucklow and Summers Virology 170:31 (1989)).

Examples of mammalian expression vectors include pWLNEO, pSV2CAT, pOG44, pXT1, pSG (Stratagene) pSVK3, PBPV, pMSG, PSVL (Pharmacia), pCDM8 (Seed, Nature 329:840 (1987)) and pMT2PC (Kaufman et al., EMBO J. 6:187 (1987)). When used in mammalian cells, the expression vector's control functions are often provided by viral regulatory elements. For example, commonly used promoters are derived from polyoma, adenovirus 2, cytomegalovirus and Simian Virus 40.

Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, adenovirus, geminivirus, and caulimovirus vectors. Non-viral vectors include plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

In addition to the regulatory control sequences discussed above, the recombinant expression vector can contain additional nucleotide sequences. For example, the recombinant expression vector can encode a selectable marker gene to identify host cells that have incorporated the vector.

Vector DNA can be introduced into prokaryotic or eukaryotic cells via conventional transformation or transfection techniques. As used herein, the terms “transformation” and “transfection” refer to a variety of art-recognized techniques for introducing foreign nucleic acids (e.g., DNA and RNA) into a host cell, including calcium phosphate or calcium chloride co-precipitation, DEAE-dextran-mediated transfection, lipofection, electroporation, microinjection, DNA-loaded liposomes, lipofectamine-DNA complexes, cell sonication, gene bombardment using high velocity microprojectiles, and viral-mediated transfection. Suitable methods for transforming or transfecting host cells can be found in Sambrook et al., Molecular Cloning: A Laboratory Manual 2nd Ed. (Cold Spring Harbor, N.Y., 1989), and other laboratory manuals.

If stable integration is desired, often only a small fraction of cells (in particular, mammalian cells) integrate the foreign DNA into their genome. In order to identify and select integrants, a nucleic acid that encodes a selectable marker (e.g., resistance to antibiotics) can be introduced into the host cells along with the nucleic acid of interest. Preferred selectable markers include those that confer resistance to drugs, such as G418, hygromycin and methotrexate. Nucleic acids encoding a selectable marker can be introduced into a host cell on the same vector as that comprising the nucleic acid of interest or can be introduced on a separate vector. Cells stably transfected with the introduced nucleic acid can be identified by drug selection (e.g., cells that have incorporated the selectable marker gene will survive, while the other cells die).

Polypeptides and fragments of the invention can be modified for in vivo use by the addition, at the amino- and/or carboxyl-terminal ends, of a blocking agent to facilitate survival of the relevant polypeptide in vivo. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. This can be done either chemically during the synthesis of the peptide or by recombinant DNA technology by methods familiar to artisans of average skill. Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the peptides can be covalently or noncovalently coupled to pharmaceutically acceptable “carrier” proteins prior to administration.

Another embodiment of the invention relates to homologs of the polypeptides of the invention that are peptidomimetic compounds that are designed based upon the amino acid sequences of the functional polypeptide fragments. Peptidomimetic compounds are synthetic compounds having a three-dimensional conformation (i.e., a “peptide motif”) that is substantially the same as the three-dimensional conformation of a selected-peptide. The peptide motif provides the peptidomimetic compound with the ability to enhance angiogenesis in a manner qualitatively identical to that of the functional fragment from which the peptidomimetic was derived. Peptidomimetic compounds can have additional characteristics that enhance their therapeutic utility, such as increased cell permeability and prolonged biological half-life.

The peptidomimetics typically have a backbone that is partially or completely non-peptide, but with side groups that are identical to the side groups of the amino acid residues that occur in the peptide on which the peptidomimetic is based. Several types of chemical bonds, e.g., ester, thioester, thioamide, retroamide, reduced carbon A, dimethylene and ketomethylene bonds, are known in the art to be generally useful substitutes for peptide bonds in the construction of protease-resistant peptidomimetics.

In one embodiment, the inhibitors of the invention (e.g., small molecules, polynucleotides, polypeptides, or homologs thereof) are administered directly to the subject. Generally, the compounds of the invention will be suspended in a pharmaceutically-acceptable carrier (e.g., physiological saline) and administered orally, topically, or by intravenous infusion, or injected subcutaneously, intramuscularly, intrathecally, intraperitoneally, intrarectally, intravaginally, intranasally, intragastrically, intratracheally, or intrapulmonarily. The inhibitors can be delivered directly to the site of the disease or disorder (e.g., the ischemic tissue), such as the lower limb (e.g., the foot or toes) or a tissue (e.g., in the heart or brain) that is affected by a myocardial or cerebral infarct. In one embodiment, the inhibitors can be delivered by limb perfusion, (optionally, isolated limb perfusion of a leg and/or arm; see, e.g. Arruda et al., Blood 105:3458 (2005)). In a further embodiment, the inhibitors can be delivered into blood vessels upstream of the ischemic tissue. In another embodiment, the inhibitors can be delivered directly to one or more muscles in or near the ischemic tissue.

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100.0 μg/kg. Wide variations in the needed dosage are to be expected in view of the variety of inhibitors available and the differing efficiencies of various routes of administration. For example, oral administration would be expected to require higher dosages than administration by i.v. injection. Variations in these dosage levels can be adjusted using standard empirical routines for optimization as is well understood in the art. Administrations can be single or multiple (e.g., 2-, 3-, 4-, 6-, 8-, 10-; 20-, 50-, 100-, 150-, or more fold). Encapsulation of the inhibitor in a suitable delivery vehicle (e.g., polymeric microparticles or implantable devices) may increase the efficiency of delivery, particularly for oral delivery.

According to certain embodiments, the inhibitors can be targeted to specific cells or tissues in vivo. Targeting delivery vehicles, including liposomes and viral vector systems are known in the art. For example, a liposome can be directed to a particular target cell or tissue by using a targeting agent, such as an antibody, soluble receptor or ligand, incorporated with the liposome, to target a particular cell or tissue to which the targeting molecule can bind. Targeting liposomes are described, for example, in Ho et al., Biochemistry 25:5500 (1986); Ho et al., J. Biol. Chem. 262:13979 (1987); Ho et al., J. Biol. Chem. 262:13973 (1987); and U.S. Pat. No. 4,957,735 to Huang et al., each of which is incorporated herein by reference in its entirety). Enveloped viral vectors can be modified to deliver a nucleic acid molecule to a target cell by modifying or substituting an envelope protein such that the virus infects a specific cell type. In adenoviral vectors, the gene encoding the attachment fibers can be modified to encode a protein domain that binds to a cell-specific receptor. Herpesvirus vectors naturally target the cells of the central and peripheral nervous system. Alternatively, the route of administration can be used to target a specific cell or tissue. For example, intracoronary administration of an adenoviral vector has been shown to be effective for the delivery of a gene to cardiac myocytes (Maurice et al., J. Clin. Invest. 104:21 (1999)). Intravenous delivery of cholesterol-containing cationic liposomes has been shown to preferentially target pulmonary tissues (Liu et al., Nature Biotechnol. 15:167 (1997)), and effectively mediate transfer and expression of genes in vivo. Other examples of successful targeted in vivo delivery of nucleic acid molecules are known in the art. Finally, a recombinant nucleic acid molecule can be selectively (i.e., preferentially, substantially exclusively) expressed in a target cell by selecting a transcription control sequence, and preferably, a promoter, which is selectively induced in the target cell and remains substantially inactive in non-target cells.

### III. SCREENING ASSAYS AND ANIMAL MODELS

The identification of a regulatory role for LAR in angiogenesis and arteriogenesis provides targets that can be used to screen for agents that regulate angiogenesis as well as models for studying the process of angiogenesis in vitro or in animals.

One aspect of the invention relates to a method of identifying a compound that regulates angiogenesis and/or arteriogenesis, comprising determining the expression and/or activity of LAR in the presence and absence of a test compound, and selecting a compound that increases or decreases the level of expression and/or activity of LAR relative to the level in the absence of the compound, thereby identifying the compound as a compound that regulates angiogenesis and/or arteriogenesis.

The assay may be a cell-based or cell-free assay. In one embodiment, the cell may be a primary cell, e.g., an endothelial cell. In another embodiment, the cell is from a cell line, e.g., an endothelial cell line. Endothelial cells and cell lines include, without limitation, HUVEC, HCEC, HGEC, HMEC-1, HUV-ST, ECY304, ECV304, and EA.hy926. The cell may be contacted with the compound in vitro (e.g., in a culture dish) or in an animal (e.g., a transgenic animal or an animal model). In one embodiment, the detected increase or decrease in expression and/or activity is statistically significant, e.g., at least p<0.05, e.g., p<0.01, 0.005, or 0.001. In another embodiment, the detected increase or decrease is at least about 10%, 20%, 30%, 40%, 50%, 60&, 70%, 80%, 90%, 100% or more.

Any desired end-point can be detected in a screening assay, e.g., binding to the LAR polypeptide, gene or RNA, modulation of the activity of LAR, modulation of angiogenesis-related pathways, and/or interference with binding by a known regulator of LAR. Methods of detecting the foregoing activities are known in the art and include the methods disclosed herein. Angiogenesis assays include, for example, induction of vascularization of the chick chorioallantoic membrane or induction of vascular endothelial cell migration as described in Ribatta et al., Intl. J. Dev. Biol., 40:1189 (1999) and Li et al., Clin. Exp. Metastasis, 17:423 (1999), respectively, and those described in McCarty et al., Intl. J. Oncol. 21:5 (2002), Akhtar et al., Clin. Chem. 49:32 (2003), and Yamashita et al., J. Biol. Chem. 269:1995 (1994). Phosphatase assays for LAR activity include, for example, assays disclosed in U.S. Pat. Nos. 7,727,752, 7,582,461, and commercially available tyrosine phosphatase assay kits.

Any compound of interest can be screened according to the present invention. Suitable test compounds include organic and inorganic molecules. Suitable organic molecules can include but are not limited to small molecules (compounds less than about 1000 Daltons), polypeptides (including enzymes, antibodies, and Fab′ fragments), carbohydrates, lipids, coenzymes, and nucleic acid molecules (including DNA, RNA, and chimerics and analogs thereof) and nucleotides and nucleotide analogs. In particular embodiments, the compound is an antisense nucleic acid, an siRNA, or a ribozyme that inhibits production of a LAR polypeptide.

Further, the methods of the invention can be practiced to screen a compound library, e.g., a small molecule library, a combinatorial chemical compound library, a polypeptide library, a cDNA library, a library of antisense nucleic acids, and the like, or an arrayed collection of compounds such as polypeptide and nucleic acid arrays.

In one representative embodiment, the invention provides methods of screening test compounds to identify a test compound that binds to LAR or a functional fragment thereof. Compounds that are identified as binding to the polypeptide or functional fragment can be subject to further screening (e.g., for modulation of angiogenesis) using the methods described herein or other suitable techniques.

Also provided are methods of screening compounds to identify those that modulate the activity of LAR or a functional fragment thereof. The term “modulate” is intended to refer to compounds that enhance (e.g., increase) or inhibit (e.g., reduce) the activity of the polypeptide (or functional fragment). For example, the interaction of LAR or functional fragment with a binding partner can be evaluated. As another alternative, physical methods, such as NMR, can be used to assess biological function. Activity of LAR or a functional fragment can be evaluated by any method known in the art, including the methods disclosed herein.

Compounds that are identified as modulators of activity can optionally be further screened using the methods described herein (e.g., for binding to a LAR polypeptide or functional fragment, polynucleotide or RNA, modulation of angiogenesis, and the like). The compound can directly interact with the LAR polypeptide or functional fragment, polynucleotide or mRNA and thereby modulate its activity. Alternatively, the compound can interact with any other polypeptide, nucleic acid or other molecule as long as the interaction results in a modulation of the activity of LAR.

As another aspect, the invention provides a method of identifying compounds that modulate angiogenesis. In one representative embodiment, the method comprises contacting a LAR polypeptide or functional fragment thereof with a test compound; and detecting whether the test compound binds to the polypeptide or functional fragment and/or modulates the activity of the polypeptide or fragment. In another exemplary embodiment, the method comprises introducing a test compound into a cell that comprises the LAR polypeptide or functional fragment; and detecting whether the compound binds to the polypeptide or functional fragment and/or modulates the activity of the polypeptide or functional fragment in the cell. The LAR polypeptide can be endogenously produced in the cell. Alternatively or additionally, the cell can be modified to comprise an isolated polynucleotide encoding, and optionally overexpressing, the LAR polypeptide or functional fragment thereof.

The screening assay can be a cell-based or cell-free assay. Further, the LAR polypeptide (or functional fragment thereof) or polynucleotide can be free in solution, affixed to a solid support, expressed on a cell surface, or located within a cell.

With respect to cell-free binding assays, test compounds can be synthesized or otherwise affixed to a solid substrate, such as plastic pins, glass slides, plastic wells, and the like. For example, the test compounds can be immobilized utilizing conjugation of biotin and streptavidin by techniques well known in the art. The test compounds are contacted with the LAR polypeptide or functional fragment thereof and washed. Bound polypeptide can be detected using standard techniques in the art (e.g., by radioactive or fluorescence labeling of the polypeptide or functional fragment, by ELISA methods, and the like).

Alternatively, the target can be immobilized to a solid substrate and the test compounds contacted with the bound polypeptide or functional fragment thereof. Identifying those test compounds that bind to and/or modulate LAR or a functional fragment thereof can be carried out with routine techniques. For example, the test compounds can be immobilized utilizing conjugation of biotin and streptavidin by techniques well known in the art. As another illustrative example, antibodies reactive with the LAR polypeptide or functional fragment can be bound to the wells of the plate, and the polypeptide trapped in the wells by antibody conjugation. Preparations of test compounds can be incubated in the polypeptide (or functional fragment)-presenting wells and the amount of complex trapped in the well can be quantitated.

In another representative embodiment, a fusion protein can be provided which comprises a domain that facilitates binding of the LAR polypeptide to a matrix. For example, glutathione-S-transferase fusion proteins can be adsorbed onto glutathione sepharose beads (Sigma Chemical, St. Louis, Mo.) or glutathione derivatized microtitre plates, which are then combined with cell lysates (e.g., ³⁵S-labeled) and the test compound, and the mixture incubated under conditions conducive to complex formation (e.g., at physiological conditions for salt and pH). Following incubation, the beads are washed to remove any unbound label, and the matrix immobilized and radiolabel detected directly, or in the supernatant after the complexes are dissociated. Alternatively, the complexes can be dissociated from the matrix, separated by SDS-PAGE, and the level of LAR polypeptide or functional fragment thereof found in the bead fraction quantitated from the gel using standard electrophoretic techniques.

Another technique for compound screening provides for high throughput screening of compounds having suitable binding affinity to the polypeptide of interest, as described in published PCT application WO84/03564. In this method, a large number of different small test compounds are synthesized on a solid substrate, such as plastic pins or some other surface. The test compounds are reacted with the LAR polypeptide or functional fragment thereof and washed. Bound polypeptide is then detected by methods well known in the art. Purified polypeptide or a functional fragment can also be coated directly onto plates for use in the aforementioned drug screening techniques. Alternatively, non-neutralizing antibodies can be used to capture the peptide and immobilize it on a solid support.

With respect to cell-based assays, any suitable cell can be used, including bacteria, yeast, insect cells (e.g., with a baculovirus expression system), avian cells, mammalian cells, or plant cells. In exemplary embodiments, the assay is carried out in a cell line that naturally expresses the polynucleotide or produces the polypeptide, e.g., endothelial cells. Further, in other embodiments, it is desirable to use nontransformed cells (e.g., primary cells) as transformation may alter the function of the polypeptide.

The screening assay can be used to detect compounds that bind to or modulate the activity of the native LAR polypeptide (e.g., polypeptide that is normally produced by the cell). Alternatively, the cell can be modified to express (e.g., overexpress) a recombinant LAR polypeptide or functional fragment thereof. According to this embodiment, the cell can be transiently or stably transformed with a polynucleotide encoding LAR or a functional fragment thereof, but is preferably stably transformed, for example, by stable integration into the genome of the organism or by expression from a stably maintained episome (e.g., Epstein Barr Virus derived episomes). In another embodiment, a polynucleotide encoding a reporter molecule can be linked to a regulatory element of the polynucleotide encoding LAR and used to identify compounds that modulate expression of the polypeptide.

In a cell-based assay, the compound to be screened can interact directly with the LAR polypeptide or functional fragment thereof (i.e., bind to it) and modulate the activity thereof. Alternatively, the compound can be one that modulates LAR polypeptide activity (or the activity of a functional fragment) at the nucleic acid level. To illustrate, the compound can modulate transcription of the gene (or transgene), modulate the accumulation of mRNA (e.g., by affecting the rate of transcription and/or turnover of the mRNA), and/or modulate the rate and/or amount of translation of the mRNA transcript.

As a further type of cell-based binding assay, the LAR polypeptide or functional fragment thereof can be used as a “bait protein” in a two-hybrid or three-hybrid assay (see, e.g., U.S. Pat. No. 5,283,317; Zervos et al., Cell 72:223 (1993); Madura et al., J. Biol. Chem. 268:12046 (1993); Bartel et al., Biotechniques 14:920 (1993); Iwabuchi et al., Oncogene 8:1693 (1993); and PCT publication WO94/10300), to identify other polypeptides that bind to or interact with the polypeptide of the invention or functional fragment thereof.

The two-hybrid system is based on the modular nature of most transcription factors, which consist of separable DNA-binding and activation domains. Briefly, the assay utilizes two different DNA constructs. In one construct, the polynucleotide that encodes the LAR polypeptide or functional fragment thereof is fused to a nucleic acid encoding the DNA binding domain of a known transcription factor (e.g., GAL-4). In the other construct, a DNA sequence, optionally from a library of DNA sequences, that encodes an unidentified protein (“prey” or “sample”) is fused to a nucleic acid that codes for the activation domain of the known transcription factor. If the “bait” and the “prey” proteins are able to interact in vivo, forming a complex, the DNA-binding and activation domains of the transcription factor are brought into close proximity. This proximity allows transcription of a reporter sequence (e.g., LacZ), which is operably linked to a transcriptional regulatory site responsive to the transcription factor. Expression of the reporter can be detected and cell colonies containing the functional transcription factor can be isolated and used to obtain the nucleic acid encoding the polypeptide that exhibited binding to the LAR polypeptide or functional fragment.

As another cell-based assay, the invention provides a method of screening a compound for modulation of angiogenesis. In particular embodiments, the cell comprises an isolated polynucleotide encoding the LAR polypeptide or functional fragment thereof. According to this embodiment, it is preferred that the isolated polynucleotide encoding the polypeptide or functional fragment is stably incorporated into the cell (i.e., by stable integration into the genome of the organism or by expression from a stably maintained episome such as Epstein Barr Virus derived episomes).

Screening assays can also be carried out in vivo in animals. Thus, as still a further aspect, the invention provides a transgenic non-human animal comprising an isolated polynucleotide encoding a LAR polypeptide or functional fragment thereof, which can be produced according to methods well-known in the art. The transgenic non-human animal can be from any species, including avians and non-human mammals. According to this aspect of the invention, suitable non-human mammals include mice, rats, rabbits, guinea pigs, goats, sheep, pigs, and cattle. Suitable avians include chickens, ducks, geese, quail, turkeys, and pheasants.

The polynucleotide encoding the LAR polypeptide or functional fragment can be stably incorporated into cells within the transgenic animal (typically, by stable integration into the genome or by stably maintained episomal constructs). It is not necessary that every cell contain the transgene, and the animal can be a chimera of modified and unmodified cells, as long as a sufficient number of cells comprise and express the polynucleotide encoding the polypeptide or functional fragment so that the animal is a useful screening tool.

Exemplary methods of using the transgenic non-human animals of the invention for in vivo screening of compounds that modulate angiogenesis and/or the activity of LAR comprise administering a test compound to a transgenic non-human animal (e.g., a mammal such as a mouse) comprising an isolated polynucleotide encoding a LAR polypeptide or functional fragment thereof stably incorporated into the genome and detecting whether the test compound modulates angiogenesis and/or polypeptide activity (or the activity of a functional fragment).

It is known in the art how to measure these responses in vivo. Illustrative approaches include observation of changes that can be studied by gross examination (e.g., formation of tubules and blood vessels), histopathology, cell markers, and enzymatic activity. Because of the potential role of LAR phosphatase in diabetes and the frequency of vascular complications in diabetics, in vivo assays of vascular function are relevant. These include: 1) measurement of vascular stiffness/compliance, made by ultrasound and other imaging modalities; 2) measurement of vascular flow reserve and other indicators of microvascular function; and 3) ophthalmic examination for retinal vascular disease including the proliferative retinopathy found in diabetes.

The transgenic non-human animals of the invention also can be used to study the processes of angiogenesis and arteriogenesis and the effect of LAR on these processes and disorders associated with these processes. Animals that overexpress LAR or do not express LAR can be created. These transgenic animals can be exposed to angiogenic or arteriogenic signals and the response of cells can be observed. In another example, the transgenic animal can be crossed with animal models of disorders associated with angiogenesis and/or arteriogenesis, such as diabetes and cardiovascular diseases, to observe the effect of LAR expression or loss of LAR expression on the disorder.

Methods of making transgenic animals are known in the art. DNA or RNA constructs can be introduced into the germ line of an avian or mammal to make a transgenic animal. For example, one or several copies of the construct can be incorporated into the genome of an embryo by standard transgenic techniques.

In an exemplary embodiment, a transgenic non-human animal is produced by introducing a transgene into the germ line of the non-human animal. Transgenes can be introduced into embryonal target cells at various developmental stages. Different methods are used depending on the stage of development of the embryonal target cell. The specific line(s) of any animal used should, if possible, be selected for general good health, good embryo yields, good pronuclear visibility in the embryo, and good reproductive fitness.

Introduction of the transgene into the embryo can be accomplished by any of a variety of means known in the art such as microinjection, electroporation, lipofection, or a viral vector. For example, the transgene can be introduced into a mammal by microinjection of the construct into the pronuclei of the fertilized mammalian egg(s) to cause one or more copies of the construct to be retained in the cells of the developing mammal(s). Following introduction of the transgene construct into the fertilized egg, the egg can be incubated in vitro for varying amounts of time, or reimplanted into the surrogate host, or both. One common method is to incubate the embryos in vitro for about 1-7 days, depending on the species, and then reimplant them into the surrogate host.

The progeny of the transgenically manipulated embryos can be tested for the presence of the construct by Southern blot analysis of a segment of tissue. An embryo having one or more copies of the exogenous cloned construct stably integrated into the genome can be used to establish a permanent transgenic animal line.

Transgenically altered animals can be assayed after birth for the incorporation of the construct into the genome of the offspring. This can be done by hybridizing a probe corresponding to the polynucleotide sequence coding for the polypeptide or a segment thereof onto chromosomal material from the progeny. Those progeny found to contain at least one copy of the construct in their genome are grown to maturity.

Methods of producing transgenic avians are also known in the art, see, e.g., U.S. Pat. No. 5,162,215.

In particular embodiments, to create an animal model in which the activity or expression of LAR is decreased, it is desirable to inactivate, replace or knock-out the endogenous gene encoding the polypeptide by homologous recombination with a transgene using embryonic stem cells. In this context, a transgene is meant to refer to heterologous nucleic acid that upon insertion within or adjacent to the gene results in a decrease or inactivation of gene expression or polypeptide amount or activity.

A knock-out of a gene means an alteration in the sequence of a gene that results in a decrease of function of the gene, preferably such that the gene expression or polypeptide amount or activity is undetectable or insignificant. Knock-outs as used herein also include conditional knock-outs, where alteration of the gene can occur upon, for example, exposure of the animal to a substance that promotes gene alteration (e.g., tetracycline or ecdysone), introduction of an enzyme that promotes recombination at a gene site (e.g., Cre in the Cre-lox system), or other method for directing the gene alteration postnatally. Knock-out animals may be prepared using methods known to those of skill in the art. See, for example, Hogan, et al. (1986) Manipulating the Mouse Embryo: A Laboratory Manual, Cold Spring Harbor Laboratory Press, Cold Spring Harbor, N.Y.

A knock-out construct is a nucleic acid sequence, such as a DNA or RNA construct, which, when introduced into a cell, results in suppression (partial or complete) of expression of a polypeptide encoded by endogenous DNA in the cell. A knock-out construct as used herein may include a construct containing a first fragment from the 5′ end of the gene encoding LAR, a second fragment from the 3′ end of the gene and a DNA fragment encoding a selectable marker positioned between the first and second fragments. It should be understood by the skilled artisan that any suitable 5′ and 3′ fragments of a gene may be used as long as the expression of the corresponding gene is partially or completely suppressed by insertion of the transgene. Suitable selectable markers include, but are not limited to, neomycin, puromycin and hygromycin. In addition, the construct may contain a marker, such as diphtheria toxin A or thymidine kinase, for increasing the frequency of obtaining correctly targeted cells. Suitable vectors include, but are not limited to, pBLUESCRIPT, pBR322, and pGEM7.

Alternatively, a knock-out construct may contain RNA molecules such as antisense RNA, siRNA, and the like to decrease the expression of a gene encoding LAR. Typically, for stable expression the RNA molecule is placed under the control of a promoter. The promoter may be regulated, if deficiencies in the protein of interest may lead to a lethal phenotype, or the promoter may drive constitutive expression of the RNA molecule such that the gene of interest is silenced under all conditions of growth. While homologous recombination between the knock-out construct and the gene of interest may not be necessary when using an RNA molecule to decrease gene expression, it may be advantageous to target the knock-out construct to a particular location in the genome of the host organism so that unintended phenotypes are not generated by random insertion of the knock-out construct.

The knock-out construct may subsequently be incorporated into a viral or nonviral vector for delivery to the host animal or may be introduced into embryonic stem (ES) cells. ES cells are typically selected for their ability to integrate into and become part of the germ line of a developing embryo so as to create germ line transmission of the knock-out construct. Thus, any ES cell line that can do so is suitable for use herein. Suitable cell lines which may be used include, but are not limited to, the 129J ES cell line or the J1 ES cell line. The cells are cultured and prepared for DNA insertion using methods well-known to the skilled artisan (e.g., see Robertson (1987) In: Teratocarcinomas and Embryonic Stem Cells: A Practical Approach, E. J. Robertson, ed. IRL Press, Washington, D.C.; Bradley et al., Curr. Topics Develop. Biol. 20:357 (1986); Hogan et al., (1986) Manipulating the Mouse Embryo: A Laboratory Manual, Cold Spring Harbor Laboratory Press, Cold Spring Harbor, N.Y.).

Insertion of the knock-out construct into the ES cells may be accomplished using a variety of methods well-known in the art, including, for example, electroporation, microinjection, and calcium phosphate treatment. For insertion of the DNA or RNA sequence, the knock-out construct nucleic acids are added to the ES cells under appropriate conditions for the insertion method chosen. If the cells are to be electroporated, the ES cells and construct nucleic acids are exposed to an electric pulse using an electroporation machine (electroporator) and following the manufacturer's guidelines for use. After electroporation, the cells are allowed to recover under suitable incubation conditions. The cells are then screened for the presence of the knockout construct.

Each knock-out construct to be introduced into the cell is first typically linearized if the knock-out construct has been inserted into a vector. Linearization is accomplished by digesting the knock-out construct with a suitable restriction endonuclease selected to cut only within the vector sequence and not within the knock-out construct sequence.

Screening for cells which contain the knock-out construct (homologous recombinants) may be done using a variety of methods. For example, as described herein, cells can be processed as needed to render DNA in them available for hybridization with a nucleic acid probe designed to hybridize only to cells containing the construct. For example, cellular DNA can be probed with ³²P-labeled DNA which locates outside the targeting fragment. This technique can be used to identify those cells with proper integration of the knock-out construct. The DNA can be extracted from the cells using standard methods (e.g., see, Sambrook et al., Molecular Cloning: A Laboratory Manual 2nd Ed. (Cold Spring Harbor, N.Y., 1989)). The DNA may then be analyzed by Southern blot with a probe or probes designed to hybridize in a specific pattern to genomic DNA digested with one or more particular restriction enzymes.

Once appropriate ES cells are identified, they are introduced into an embryo using standard methods. They can be introduced using microinjection, for example. Embryos at the proper stage of development for integration of the ES cell to occur are obtained, such as by perfusion of the uterus of pregnant females. For example, mouse embryos at 3-4 days development can be obtained and injected with ES cells using a micropipet. After introduction of the ES cell into the embryo, the embryo is introduced into the uterus of a pseudopregnant female mouse. The stage of the pseudopregnancy is selected to enhance the chance of successful implantation. In mice, 2-3 days pseudopregnant females are appropriate.

Germline transmission of the knockout construct may be determined using standard methods. Offspring resulting from implantation of embryos containing the ES cells described above are screened for the presence of the desired alteration (e.g., knock-out of the LAR polypeptide). This may be done, for example, by obtaining DNA from offspring (e.g., tail DNA) to assess for the knock-out construct, using known methods (e.g., Southern analysis, dot blot analysis, PCR analysis). See, for example, Sambrook et al., Molecular Cloning: A Laboratory Manual 2nd Ed. (Cold Spring Harbor, N.Y., 1989). Offspring identified as chimeras may be crossed with one another to produce homozygous knock-out animals.

Mice are often used as animal models because they are easy to house, relatively inexpensive, and easy to breed. However, other knock-out animals may also be made in accordance with the present invention such as, but not limited to, monkeys, cattle, sheep, pigs, goats, horses, dogs, cats, guinea pigs, rabbits and rats. Accordingly, appropriate vectors and promoters well-known in the art may be selected and used to generate a transgenic animal deficient in expression of LAR.

In another embodiment, animal models may be created using animals that are not transgenic. For example, tumor models (e.g., created by delivering tumorigenic cells into immunocompromised animals) can be used to study the effects of regulators of angiogenesis on tumor growth and metastasis. In another example, tumorigenic cells that overexpress or underexpress LAR can be delivered to an animal under conditions in which tumors develop from the cells. Tumor growth in the animals can be compared to tumor growth in animals containing cells that do not overexpress or underexpress the polypeptide.

### IV. PHARMACEUTICAL COMPOSITIONS

As a further aspect, the invention provides pharmaceutical formulations and methods of administering the same to achieve any of the therapeutic effects (e.g., stimulation of angiogenesis) discussed above. The pharmaceutical formulation may comprise any of the reagents discussed above in a pharmaceutically acceptable carrier, e.g., an antibody against LAR, an antisense oligonucleotide, an siRNA molecule, a ribozyme, an aptamer, a peptidomimetic, a polynucleotide encoding an inhibitor of LAR, a small molecule, or any other compound that modulates the activity of LAR, including compounds identified by the screening methods described herein.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

The compounds of the invention can be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the compound (including the physiologically acceptable salts thereof) is typically admixed with, inter alia, an acceptable carrier. The carrier can be a solid or a liquid, or both, and is preferably formulated with the compound as a unit-dose formulation, for example, a tablet, which can contain from 0.01 or 0.5% to 95% or 99% by weight of the compound. One or more compounds can be incorporated in the formulations of the invention, which can be prepared by any of the well-known techniques of pharmacy.

A further aspect of the invention is a method of treating subjects in vivo, comprising administering to a subject a pharmaceutical composition comprising a compound of the invention in a pharmaceutically acceptable carrier, wherein the pharmaceutical composition is administered in a therapeutically effective amount. Administration of the compounds of the present invention to a human subject or an animal in need thereof can be by any means known in the art for administering compounds.

The formulations of the invention include those suitable for oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intrathecal, and inhalation administration, administration to the liver by intraportal delivery, as well as direct organ injection (e.g., into the liver, into a limb, into the brain for delivery to the central nervous system, into the pancreas, or into a tumor or the tissue surrounding a tumor). The most suitable route in any given case will depend on the nature and severity of the condition being treated and on the nature of the particular compound which is being used. In some embodiments, it may be desirable to deliver the formulation locally to avoid any side effects associated with systemic administration. For example, local administration can be accomplished by direct injection at the desired treatment site, by introduction intravenously at a site near a desired treatment site (e.g., into a vessel that feeds a treatment site), or directly to the wall of a vessel (e.g., using a drug delivery catheter such as a balloon catheter). In some embodiments, the formulation can be delivered locally to ischemic tissue. In certain embodiments, the formulation can be a slow release formulation, e.g., in the form of a slow release depot.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Compounds can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose container, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a compound of the invention, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 1 mg to about 10 grams of the compound or salt. When the compound or salt is substantially water-insoluble, a sufficient amount of emulsifying agent which is pharmaceutically acceptable can be employed in sufficient quantity to emulsify the compound or salt in an aqueous carrier. One such useful emulsifying agent is phosphatidyl choline.

Formulations suitable for rectal administration are preferably presented as unit dose suppositories. These can be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the compound. Suitable formulations comprise citrate or bis\tris buffer (pH 6) or ethanol/water and contain from 0.1 to 0.2M of the compound.

The compound can alternatively be formulated for nasal administration or otherwise administered to the lungs of a subject by any suitable means, e.g., administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the compound can likewise be produced with any solid particulate medicament aerosol generator, by techniques known in the pharmaceutical art.

Alternatively, one can administer the compound in a local rather than systemic manner, for example, in a depot or sustained-release formulation.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same can be incorporated into lipid vesicles. In such an instance, due to the water solubility of the compound or salt, the compound or salt will be substantially entrained within the hydrophilic center or core of the liposomes. The lipid layer employed can be of any conventional composition and can either contain cholesterol or can be cholesterol-free. When the compound or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the compounds disclosed herein or salts thereof, can be lyophilized to produce a lyophilizate which can be reconstituted with a pharmaceutically acceptable carrier, such as water, to regenerate a liposomal suspension.

In the case of water-insoluble compounds, a pharmaceutical composition can be prepared containing the water-insoluble compound, such as for example, in an aqueous base emulsion. In such an instance, the composition will contain a sufficient amount of pharmaceutically acceptable emulsifying agent to emulsify the desired amount of the compound. Particularly useful emulsifying agents include phosphatidyl cholines and lecithin.

In particular embodiments, the compound is administered to the subject in a therapeutically effective amount, as that term is defined above. Dosages of pharmaceutically active compounds can be determined by methods known in the art, see, e.g., Remington's Pharmaceutical Sciences (Maack Publishing Co., Easton, Pa.). The therapeutically effective dosage of any specific compound will vary somewhat from compound to compound, and patient to patient, and will depend upon the condition of the patient and the route of delivery. As a general proposition, a dosage from about 0.1 to about 50 mg/kg will have therapeutic efficacy, with all weights being calculated based upon the weight of the compound, including the cases where a salt is employed. Toxicity concerns at the higher level can restrict intravenous dosages to a lower level such as up to about 10 mg/kg, with all weights being calculated based upon the weight of the compound, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Particular dosages are about 1 μmol/kg to 50 μmol/kg, and more particularly to about 22 μmol/kg and to 33 μmol/kg of the compound for intravenous or oral administration, respectively.

In particular embodiments of the invention, more than one administration (e.g., two, three, four, or more administrations) can be employed over a variety of time intervals (e.g., hourly, daily, weekly, monthly, etc.) to achieve therapeutic effects.

The present invention finds use in veterinary and medical applications as well as in research. Suitable subjects include both avians and mammals, with mammals being preferred. The term “avian” as used herein includes, but is not limited to, chickens, ducks, geese, quail, turkeys, and pheasants. The term “mammal” as used herein includes, but is not limited to, humans, bovines, ovines, caprines, equines, felines, canines, lagomorphs, etc. Human subjects include neonates, infants, juveniles, and adults. In other embodiments, the subject is an animal model of ischemia. In certain embodiments, the subject has or is at risk for diabetes (e.g., Type I or Type II diabetes). In other embodiments, the subject has cardiovascular disease or has experienced ischemia or stroke. In still other embodiments, the subject is at risk for ischemia. In further embodiments, the subject is in need of increase angiogenesis and/or arteriogenesis.

The present invention is more particularly described in the following examples that are intended as illustrative only since numerous modifications and variations therein will be apparent to those skilled in the art.

### Example 1

### Experimental Methods

Materials:

Recombinant human IGF-1 and VEGF were purchased from R&D Systems (Minneapolis, Minn.). Antibodies for eNOS and phospho eNOS (Ser1177), Akt, phospho Akt (Ser 473), ERK1/2, phospho ERK1/2 (Thr202/Tyr204), VEGFR2 and VEGFR3 were purchased from Cell Signaling Technology (Beverly, Mass.). Anti-IGF-1 receptor and β-actin antibodies were from Santa Cruz Biotechnology (Santa Cruz, Calif.). Monoclonal antibody against phosphotyrosine (4G10) was obtained from Millipore (Billerica, Mass.). Two different anti-LAR antibodies, anti-LAR monoclonal (catalog number 610350, BD Transduction Laboratories) and goat anti-LAR polyclonal (sc-1119, Santa Cruz Biotechnology), were used in this study. The monoclonal antibody was raised against an epitope corresponding to amino acids 24-194 of human LAR and recognizes the 150-kDa extracellular fragment. The goat polyclonal antibody was raised against an epitope in the COOH-terminal cytoplasmic domain of rat LAR and recognizes the 85-kDa C-terminal subunit of human LAR. Growth factor reduced Matrigel Matrix (BD Biosciences, San Jose, Calif.) was used for both cell culture and to maintain aortic rings in culture. Picropodophyllin (PPP) was purchased from Sigma (Sigma-Aldrich). B6.BKS-Lepr^{db/db} diabetic mice were purchased from The Jackson Laboratory (Bar Harbor, Me., USA). 12 week-old B6.BKS-Lepr^{db/db} (Db/Db) diabetic mice were used for lentivirus infected experiments. Mice were housed at the Association for Assessment and Accreditation of Laboratory Animal Care, and maintained in accordance with the National Research Council's Guide for the Care and Use of Laboratory Animals. All animal procedures and experiments were approved by the institutional animal care and use committee.

Cell Culture:

Human umbilical vein endothelial cells (HUVECs) were purchased from Cambrex (San Diego, Calif.) and were maintained in medium 199 (Mediatech, Inc, Herndon, Va.) containing 90 μg/ml heparin, 30 μg/ml endothelial cell growth supplements (BD Biosciences, Bedford, Mass.), 20% FBS, 50 μg/mL gentamicin, and 50 μg/mL amphotericin. Cultures were maintained at 37° C. in a humidified incubator (95% air, 5% CO₂). HUVECs, grown to 80% confluence, were growth arrested by overnight incubation in medium 199 supplemented with 0.1% serum. Growth-arrested HUVECs were treated with VEGF or IGF-1 in serum-free medium 199 for the indicated times.

Immunoprecipitation and Western Blot Analysis:

Preparation of cell lysates, immunoprecipitation and immunoblotting were performed as described (Reinmuth et al., Clin. Cancer Res. 8:3259 (2002)).

Construction of Adenoviral Vectors Expressing Full-length Human LAR and β-Galactosidase:

Adenoviral constructs encoding β-galactosidase and full-length human LAR were prepared as described previously (Reinmuth et al., Clin. Cancer Res. 8:3259 (2002)). Recombinant adenoviruses were serially amplified in human embryonic kidney 293 cells and then purified using the ViraBind adenovirus purification kit (Cell Biolabs, Inc., San Diego, Calif.).

Adenovirus Infection:

Adenoviral infection of nearly confluent HUVECs was performed with indicated multiplicity of infection in medium 199 containing 2% FBS. After 16 h incubation, the cells were quiesced overnight in medium 199 containing 0.1% FBS. Then HUVECs were treated with the indicated agonists in serum-free medium 199.

Transient Transfection with LAR siRNA:

The LAR siRNA (sc-35793, containing three siRNAs (SEQ ID NOS:1-3)) and a scrambled, non-silencing siRNA (sc-36869) were obtained from Santa Cruz Biotechnology. The siRNA duplexes were preincubated with oligofectamine 2000 (Invitrogen) according to the manufacturer's protocol, and the transfection complexes were added to HUVECs at 80% confluence in the OPTI medium (Life Technologies) and incubated for 48 h. Then HUVECs were quiesced by incubating overnight in medium 199 containing 0.1% FBS. HUVECs were treated with the indicated agonists in serum-free medium 199 and LAR expression was assessed by Western analysis.

Cell Migration:

HUVEC migration was analyzed in the scratched wound assay. HUVECs were grown to 80% confluence in 6-well plates previously labeled with a traced line. The cells were quiesced in medium 199 containing 0.1% FBS. The cell monolayers were scraped with a disposable rubber policeman to create cell-free zones. The cell monolayers were washed with serum-free medium 199 and then stimulated with IGF-1 or VEGF. EC migration was quantified measuring the length of the cell migration zone (distance between the traced line and the edges of the cell monolayer) 16 h after stimulation using a computer-assisted microscope (Nikon) at 5 distinct positions (every 5 mm).

Cell Proliferation:

AdLAR- and Adβ-gal-infected HUVECs were plated in 12-well plates at a density of 40,000 cells/well. After 24 h, the medium was replaced with endothelial cell basal medium (EBM, BD Biosciences, Bedford, Mass.), and cells were incubated for an additional 24 h. The medium was then replaced with either fresh EBM alone or EBM containing IGF-1 (100 ng/ml) or VEGF (20 ng/ml). The cells were incubated for approximately 15 h, followed by a 3 h labeling with 1 μCi/ml [³H]thymidine (Amersham Pharmacia Biotech). The DNA was precipitated with cold 10% trichloroacetic acid at 4° C. for 30 min followed by wash with an absolute ethanol. The precipitated material was resuspended in 0.5 ml of 1 M NaOH and neutralized with 1 M HCl. [³H]thymidine incorporation was determined from 500 μl aliquots using a Beckman LS6000SC Scintillation Counter.

Matrigel Morphogenesis Assay:

Growth factor-reduced Matrigel was thawed at 4° C. on ice and 300 μl/well were added to a 24-well plate prechilled at −20° C. using a cold pipette. Matrigel was allowed to polymerize for 30 min at 37° C. HUVECs were either left uninfected or were infected with AdLAR or Adβ-gal. In a separate set of experiments, HUVECs were transfected with LAR siRNA or control siRNA as described above. HUVECs (2×10⁴/well) were layered on top of the Matrigel in medium 199 and stimulated with IGF-1 or VEGF. In vitro tube formation was assessed after 5 h incubation. Cell cultures were observed under a phase contrast microscope and photographed in five different random fields (10× magnification). The mean tube length per field was quantified using NIH Image J.

Ex Vivo Angiogenesis Assay:

The angiogenesis assay was performed as described previously (Huang et al., J. Biol. Chem. 277:10760 (2002)) with some modifications. Thoracic aortas were excised from 8-12-week-old wild-type and LAR knockout male mice (DBA background) and immediately placed into cold medium 199 containing 10% FBS. Clotted blood inside the aortas was flushed with media, and the periadventitial fibroadipose tissue was removed. Aortas were then cut into cross-sectional rings of ˜1-1.5 mm in length. Rings were placed into 60-mm dishes containing 0.4 ml of cold growth factor-reduced Matrigel and then incubated at 37° C. until the Matrigel was polymerized. The aortic rings embedded in the Matrigel were overlaid with 0.5 ml of serum-free endothelial cell growth medium with or without the growth factors. The rings were maintained at 37° C. for up to 10 days with medium changes every 2 days. Vascular sprouting from the rings was examined daily under a Leica microscope (10× magnification), and digital images were obtained. Quantitative analysis of endothelial sprouting was performed using images from day 10, and sprout length was quantified using NIH Image J. The distance from the aortic ring body to the end of the vascular sprouts was measured at three distinct points per ring.

In Vivo Angiogenesis Assay:

The Matrigel plug assay was used to assess in vivo angiogenesis. 8-12-week-old wild-type and LAR^{-/-} mice (DBA background) were anesthetized with 1.5% isoflurane by inhalation and injected subcutaneously on the ventral abdomen with 500 μl Matrigel containing 500 ng/ml IGF-1, 500 ng/ml basic fibroblast growth factor and 5 U/ml heparin or basic fibroblast growth factor and heparin alone. Mice were sacrificed after 10 days and the plugs were carefully dissected from the mice. The plugs were weighed, analyzed histologically or for hemoglobin content using Drabkin hemoglobin assay kit (Sigma Chemical Co., St. Louis, Mo.).

Hindlimb Ischemia:

Hindlimb ischemia was induced in 12-16-week-old LAR^{-/-} mice and wild-type littermate controls (DBA background) by femoral artery ligation Clayton et al., Circ. Res. 103:1027 (2008)). Mice were randomized and procedures/analyses were conducted in a blinded fashion. Mice were anesthetized with 1.25% isoflurane/O₂ and the hindlimbs were depilated. The right femoral artery was exposed through a 2-mm incision and ligated with two 7-0 ligatures placed distal to the origin of the lateral caudal femoral and superficial epigastric arteries and proximal to the genu artery. The artery was transected between the sutures and separated by 2 mm. The wound was washed with sterile saline and closed, and cefazolin (50 mg/kg, im), furazolidone (topical) and pentazocine (10 mg/kg, im) were administered. Body temperature was maintained at 37.0±0.5° C. during the procedure. The above mentioned arteries from the opposite limb were not ligated and served as an internal control to calculate perfusion recovery. The procedures were approved by the University of North Carolina Institutional Animal Care and Use Committee.

Assay of Hemoglobin Content of Matrigel Plugs:

Hemoglobin levels in the Matrigel plugs correlate well with blood vessel growth (Volin et al., Am. J. Pathol. 159:1521 (2001)). The Matrigel plugs were homogenized on ice and centrifuged. Fifty μl of the supernatant were mixed with 950 μl Drabkin's reagent and the reaction was allowed to develop at room temperature for 30 min. Absorbance was read at 540 nm and hemoglobin concentration was determined by comparison to a standard curve generated using known concentrations of hemoglobin.

Quantitation of Endothelial Progenitor Cells (EPCs) by Flow Cytometry:

To quantify circulating EPC, peripheral blood (PB) was collected by cardiac puncture in PBS with 50 mmol/L EDTA. Mononuclear cells from 500 μl PB were separated by Ficoll-Paque™ PREMIUM products and incubated in ALK buffer [150 mmol/L NH₄Cl, 10 mmol/L KHCO₃, 0.1 mmol/L EDTA, pH 7.3] on ice for 10 min to lyse RBC. Cells were washed twice with Hank's balanced saline+2% FBS (HF), and counted using a hemocytometer to calculate the total PB cell number per 1 mL of blood. Cells were resuspended in incubation buffer (2% FBS, 20 mmol/L HEPES, 2 mmol/L EDTA in DMEM), blocked with anti-mouse CD16/CD32 (14-0161), and then labeled with anti-CD45-FITC (12-5821, eBioscience), anti-CD34-APC (560233, BD Pharmingen), and anti-Flk1-PE (clone Avas eBioscience) antibodies at 4° C. for 30 min. After washing twice with cold HF, cells were resuspended in cold PBS/2% FCS and placed on ice, before analysis using a FACS CyAn (CyAn ADP). To quantify Flk⁺CD34⁺ double-positive cells, the mononuclear cell fraction was gated and analyzed for the expression of CD34 and CD45. 50,000 viable cells were analyzed in each sample. Circulating EPCs were defined as cells expressing both Flk-1 and CD34.

Histology

In Vivo Angiogenesis:

For histological analysis, Matrigel plugs were formalin-fixed, paraffin-embedded, cut into 5 μm sections, and Massons trichrome-stained. In brief, sections were hydrated in distilled water, incubated in Bouin's fixative for 3 h to overnight, and then washed in running tap water until yellow color disappeared. The sections were rinsed in distilled water, and dipped in Weigert's iron hematoxylin solution for 10 min, washed in running water for 10 min, and dipped in Biebrich scarlet-acid fuchsin solution for 5 min. Sections were then rinsed, incubated in phosphomolybdic-phosphotungstic acid solution for 10 min, dipped in aniline blue solution for 3 min, rinsed, dipped in 0.5% acetic acid solution for 30 sec-1 min, and dehydrated in two changes of 95% alcohol, 100% alcohol, and xylene. The slides were mounted with Cytoseal 60 (Stephens Scientific, Kalamazoo, Mich.).

Hindlimb Ischemia:

Mice were pressure-perfused transcardially (100 mmHg) 14 days after ligation with phosphate-buffered saline (pH 7.4) containing 20 mmol/L adenosine, 10⁻⁴ mol/L papaverine and 10 U/ml heparin, followed by pressure-perfused fixation with 4% paraformaldehyde in 100 mmol/L sodium phosphate (PFA; pH 7.4) for 15 min. The hindquarters were post-fixed while shaking for 48 h in PFA, with a solution change at 24 h. The adductor muscles (medial thigh) below the femur were excised as a ˜5 mm square block extending ˜1 mm medial of the “mid-zone” of the gracilis muscle collaterals that interconnect the lateral caudal femoral artery and saphenous artery trees, to the lateral portion of the thigh. An ˜5 mm square block of the calf was excised beginning at the origin of the Achilles tendon and extending toward the knee. Blocks were rinsed in water, placed in 70% ethanol for 48 h with a solution change at 24 h and embedded in paraffin. Cross-sections (5 μm) perpendicular to the long axis of muscle fibers, capillaries and direction of the gracilis collaterals, beginning on the medial side for the adductor and on the knee-side for the calf were made. After removing the first 200 μm of the full block face, sections 250 μm apart were cut and arranged 6 per slide. Collateral lumen diameter in the anterior and posterior gracilis muscles was determined from the mid-zone of sections stained with cyano-Massons trichrome. Results reported were average values of 2 sections from digitized images analyzed with Image J, which were 250 pin apart.

Capillary density and capillary-to-muscle fiber number in the gastrocnemius muscle 14 days after ligation were determined by staining with biotinylated Griffonia Simplicifolia isolectin-1-B₄ (1:100, Vector Labs) plus Alexa Fluor 488 conjugate of streptavidin (1:50, Molecular Probes). Capillaries were identified as lectin-positive vessels with diameters <7 μm in coronal sections of the gastrocnemius muscle within a delineated area. For muscle atrophy, average muscle fiber area was determined for the fascicles within the same area. Boundaries of fibers and fascicles were outlined by autofluorescence during imaging. Capillary density was reported as average density per square micrometer and as capillary number-to-muscle fiber number ratio. Capillary density, ratio and fiber area were obtained from 2 digitized 20× fields of view (˜434 μm×330 μm) within 2 sections each of the medial and lateral heads of the gastrocnemius of the ligated and non-ligated leg and were averaged (Image J).

Cerebral Arteriography: Collateral Number and Tree Structure in the Pial Circulation:

Mice were cannulated via the descending abdominal aorta, heparinized, perfusion-cleared and maximally dilated (10 mg/ml adenosine and 4 mg/ml papaverine). The dorsal calvarium and adherent dura mater were removed to expose the pial circulation. A second catheter was placed retrogradely into the thoracic aorta, and Microfil™ (FlowTech, Inc., Carver, Mass.) with a viscosity sufficient to minimize capillary transit was infused with the aid of a stereomicroscope. PFA (4%) was applied topically, and the latex was allowed to cure. After fixation with PFA, the pial circulation was imaged (Leica MZ16FA, Leica Microsystems 6 Bannockburn, Ill.). Collaterals connecting the middle and anterior cerebral artery trees of both hemispheres were counted.

Laser Doppler Perfusion Imaging:

The mice were anesthetized with 1.25% isoflurane/O₂ anesthesia and placed on a heating pad maintained at a constant temperature of 37° C. Hair was removed with a depilatory cream. A Laser Doppler Perfusion Imager (Moor Instruments Ltd, Devon, United Kingdom) was used to measure dermal blood flow in the adductor thigh region and plantar foot of both limbs before, immediately after, and at 3, 7 and 14 days after femoral ligation. Regions of interest were drawn to anatomical landmarks. Ratios of the operated/nonoperated blood flow were used to account for differences in ambient temperature and lighting.

Immunofluorescence and Histological Analysis:

85% confluent HUVEC were fixed with 4% paraformaldehyde for 10 min and cells were permeabilized by 0.25% triton-100 for 5 min. After washing with PBS, cells were blocked by 1% BSA for 1 hour. Incubation with the primary antibody was performed for 2 hours, and with secondary antibodies conjugated to Fluorescent (Molecular Probes Inc.) for 1 hour at room temperature. For co-localization of LAR with phospho-IGF-1 β receptor, monolayer was probed with goat anti-LAR antibody and rabbit anti-phospho IGF-1 receptor antibody in blocking buffer, respectively, followed by Alexa Fluor 488 anti-rabbit antibody and Alexa Fluor 568 anti-goat antibody (Molecular Probes Inc.). All slides were stained with 4′,6-diamidino-2-phenylindole (DAPI). Confocal images were captured on a Leica SP2 confocal (Laser Scanning Microscope), using 63×1.4 N/A oil immersion objective, the 488 nm excitation wavelength of an argon laser (green), and a 543 nm HENE (red), and 361 UV laser (blue). Images were processed using Photoshop, and imported into Adobe Illustrator CS. For colocalization of CD31, CD45, and α-SM actin with LAR, at day 3 or day 21, paraffin-embedded tissue sections of adductor muscle and gastrocnemius muscle, antigen retrieval was carried out at pH 6 (BD Retrievagen A) at 95° C. for 20 min. Samples were blocked with normal goat serum for 1 hour and then incubated with either CD31 (rat polyclonal antibody, 1:50), α-SM actin (mouse monoclonal antibody (1:400)) and CD45 (rabbit polyclonal antibody, 1:50)) or anti LAR (goat polyclonal antibody, 1:50) for 1 hour at room temperature. Sections were incubated in primary antibody (in blocking buffer) overnight at 4° C. followed by 3×5 min washes with TBST. Application of secondary antibody (Alexa 488 goat anti-mouse, 1/200 or Alexa 488 goat anti-rabbit, 1/200, or application of secondary antibody, Alex 488 goat anti rat IgG, Alexa 568 donkey anti goat IgG, Molecular Probes) in a TBS solution was for 1 h at room temperature. As a control, immunohistochemistry was done without anti-LAR, CD31, α-SM actin and CD45. In none of these control experiments was a specific signal obtained. Sections were lightly counterstained with DAPI. Images at ×40 or ×20 magnification.

LAR shRNA Lentivirus:

shRNA against mouse LAR was purchased from Lenti-shRNA Core Facility (University of North Carolina at Chapel Hill). Non-targeting shRNA (Addgene 10879) was used as a control. The double-stranded hairpin loop-containing sequence of the shRNA used in this study was 5-CCGGCGCTTTGAGGTAATTGAGTTTCTCGAGAAACTCAATTACCTCAAAGCGT TTTT-3. VSV-G pseudotyped HIV-1 based LAR shRNA vectors were prepared by standard calcium phosphate transfection method in 293T cells.

Lentiviral Vector Transductions:

Unilateral hindlimb ischemia was created in 10 week-old B6.BKS-Lepr^{db/db} (Db/Db) diabetic mice as described above. The animals were anesthetized by isoflurane inhalation. An incision was made at the midline of the left hindlimb. The femoral artery and its branches were ligated, beginning from the inguinal ligament to the bifurcation of saphenous and popliteal arteries. The region between the ligatures was excised. After this, the mice were randomly separated into two groups (n=5 per group). LAR shRNA or scrambled shRNA lentivirus in saline were injected through 100 μl microsyringe. The hindlimb was gently lifted and extended to better visualize the location of the adductor and gastrocnemius muscle. The needle was inserted into the underlying muscle. Care was taken not to approach the bone. The 30 μL virus (lentiviral vectors were diluted with sterile saline to achieve a titer of 1×10⁹ u/ml) were gently and slowly multi-injected into the adductor and gastrocnemius. Viral preparations in 1-2-uL volume were injected into each point. The incision was closed with 7-0 silk sutures.

Imaging Analysis for Angiogenesis:

Measurement of the level of angiogenesis in the in vivo Matrigel plug experiment was based on the Masson's Trichrome staining, the regions of the most intense area of neovascularization were chosen for analysis. Five areas were identified for each Matrigel section. Image J was used to quantify the number of vessel-like structures in each field. Total number of microvessels containing red blood cells and number of capillaries in a high power field (20× magnifications) were counted. The mean and the SE of mean from each group were compared.

Statistical Analysis:

All results are expressed as mean±SEM. Data from in vitro studies were analyzed with one-way analysis of variance, and post hoc analysis was performed using the Newman-Keuls test. Statistical significance was accepted at P<0.05. For hindlimb ischemia studies, significance (P<0.05) was determined by two-way ANOVA followed by Dunn-Bonferonni Corrected t-tests.

### Example 2

### Increased LAR Expression Specifically Inhibits IGF-1-Induced Autophosphorylation of IGF-1Rβ, Whereas Decreased LAR Expression Enhances IGF-Induced Phosphorylation of IGF-1Rβ

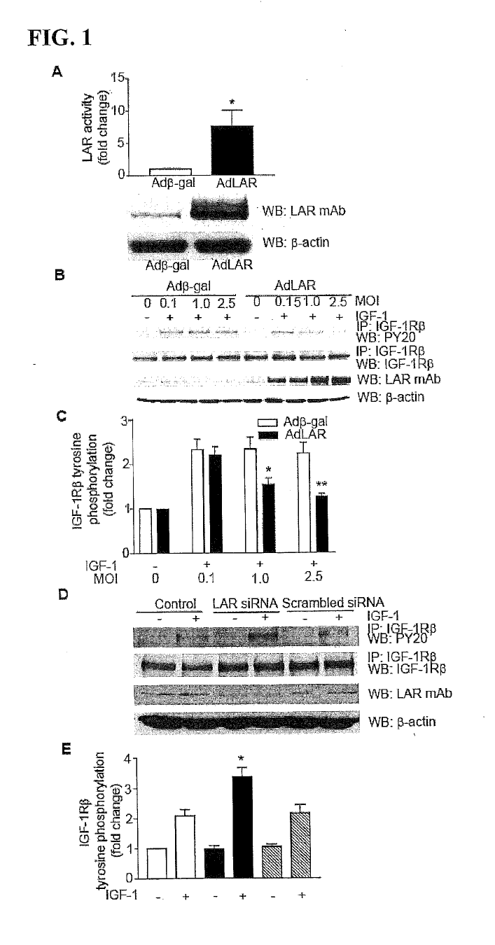

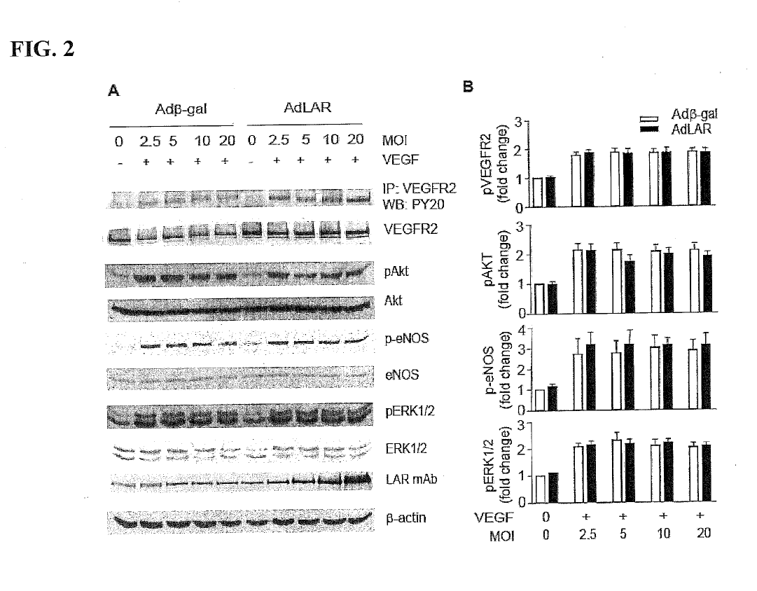

Angiogenic growth factor activation of specific receptor tyrosine kinases (RTKs) in ECs results in angiogenesis and arteriogenesis (Kappert et al., Cardiovasc. Res. 65:587 (2005)). Because both IGF-1 and VEGF play a prominent role in angiogenesis, the effect of LAR overexpression on autophosphorylation of IGF-1Rβ and VEGFR2 was examined in HUVECs treated with IGF-1 and VEGF, respectively. LAR expression and activity in HUVECs transduced with adenoviral LAR (AdLAR) were significantly higher than in cells transduced with adenoviral β-galactose (Adβ-gal) (FIG. 1A). LAR overexpression inhibited IGF-1-induced tyrosine phosphorylation of IGF-1Rβ in a dose-dependent manner, with maximum inhibition at 2.5 MOI (FIGS. 1B and 1C). Overexpression of either AdLAR or Adβ-gal had no effect on HUVEC IGF-1Rβ expression levels. In contrast to its effect on IGF-1-induced IGF-1Rβ phosphorylation, overexpression of LAR had no effect on VEGF-induced tyrosine phosphorylation of VEGFR2 in HUVECs (FIGS. 2A-2B).

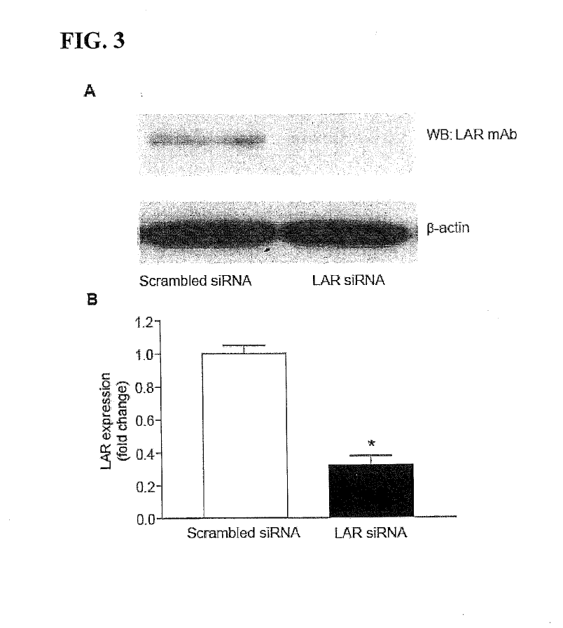

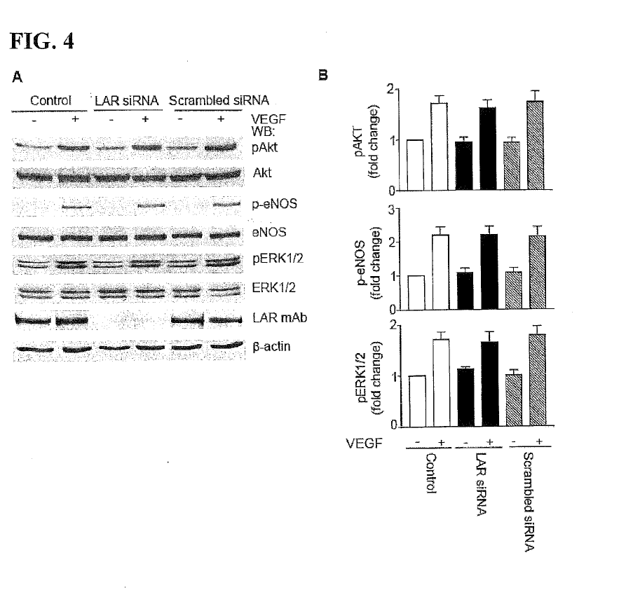

Consistent with LAR-dependent inhibition of IGF-1-induced IGF-1Rβ phosphorylation, knockdown of endogenous LAR with LAR-specific siRNA (FIGS. 3A-3B) significantly increased IGF-1Rβ activity, without an apparent effect on IGF-1Rβ expression levels, in HUVEC treated with IGF-1 (FIGS. 1D and 1E). LAR knockdown did not affect VEGF-induced activation of VEGFR2 downstream signaling moieties (FIGS. 4A-4B). These data suggest that LAR functions as specific negative regulator of IGF-1Rβ autophosphorylation in HUVECs.

### Example 3

### IGF-1 Enhances the Formation of IGF-1Rβ-LAR Complex in HUVECs

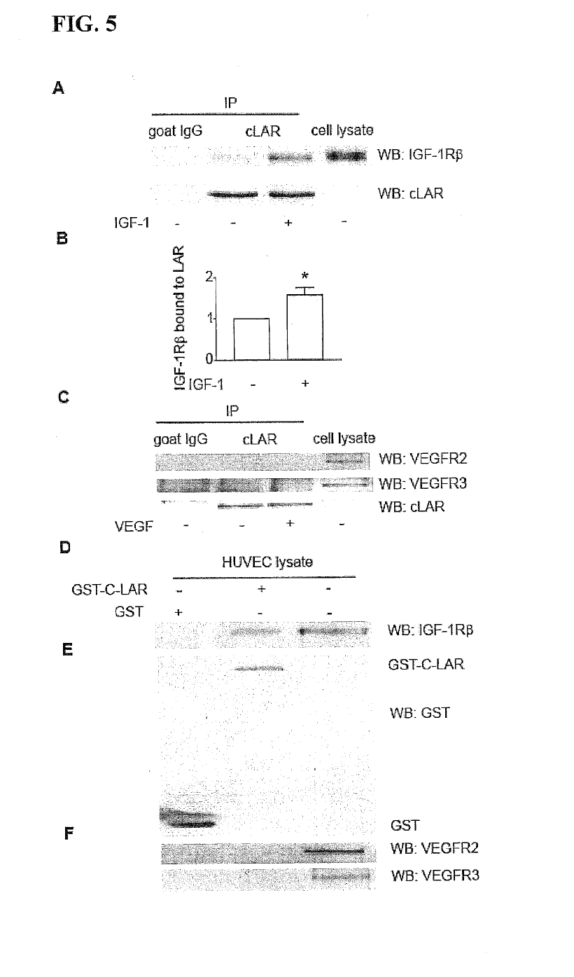

It was previously demonstrated that IGF-1 treatment increases the physical association of LAR with the IGF-1R in VSMCs (Niu et al., J. Biol. Chem. 282:19808 (2007)). Analogous experiments were performed to determine whether binding of LAR to IGF-1Rβ accounts for the negative regulation of IGF-1Rβ by this PTP in HUVECs. Cell lysates from HUVECs, treated without or with IGF-1, were immunoprecipitated with anti-cytoplasmic LAR (cLAR) antibody and Western blotting was performed with anti-IGF-1Rβ antibody. LAR co-immunoprecipitated with IGF-1Rβ in untreated HUVECs and IGF-1 treatment significantly enhanced association of LAR with IGF-1Rβ (FIGS. 5A and 5B). Consistent with the selective regulation of IGF-1Rβ, LAR did not co-immunoprecipitate with VEGFR2 or VEGFR3 either in basal state or after VEGF treatment (FIG. 5C).

To gain additional evidence for LAR-IGF-1Rβ interaction observed in vivo, it was examined whether IGF-1Rβ in HUVEC lysates binds to GST-cLAR in vitro. Western analysis revealed that cLAR binds with IGF-1Rβ in HUVEC lysates (FIGS. 5D and 5E), but not with VEGFR2 or VEGFR3 (FIG. 5F). Together with the previous data showing that LAR dephosphorylates activated IGF-1Rβ in vitro (Niu et al., J. Biol. Chem. 282:19808 (2007)), these results suggest that LAR binds to IGF-1Rβ independent of its phosphorylation status and IGF-1Rβ is a substrate for LAR in vivo.

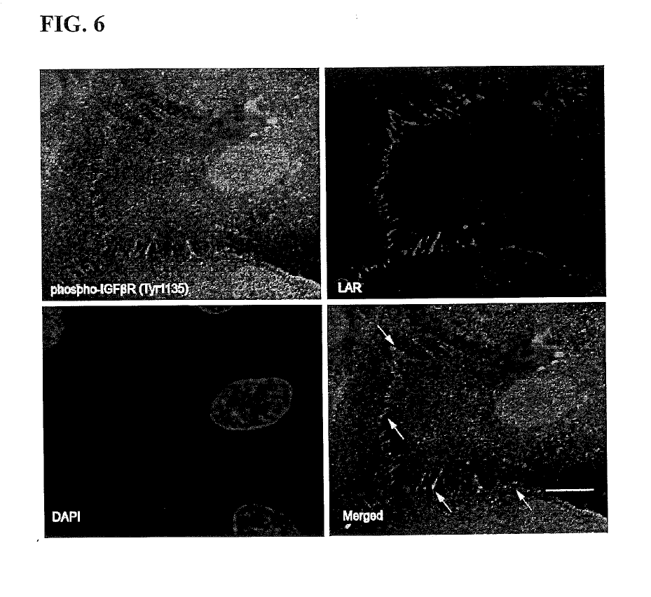

To further investigate the interaction between LAR and IGF-1Rβ, the localization of these two proteins was determined in HUVECs treated with IGF-1 by confocal immunofluorescence microscopy (FIG. 6). Superimposition of images of HUVECs immunolabeled with phospho-IGF-1Rβ (green) and LAR (red) antibodies indicates colocalization of LAR with IGF-1Rβ at the membrane as indicated by yellow staining.

### Example 4

### Increased LAR Expression Inhibits IGF-1Rβ (but Not VEGFR2) Downstream Signals

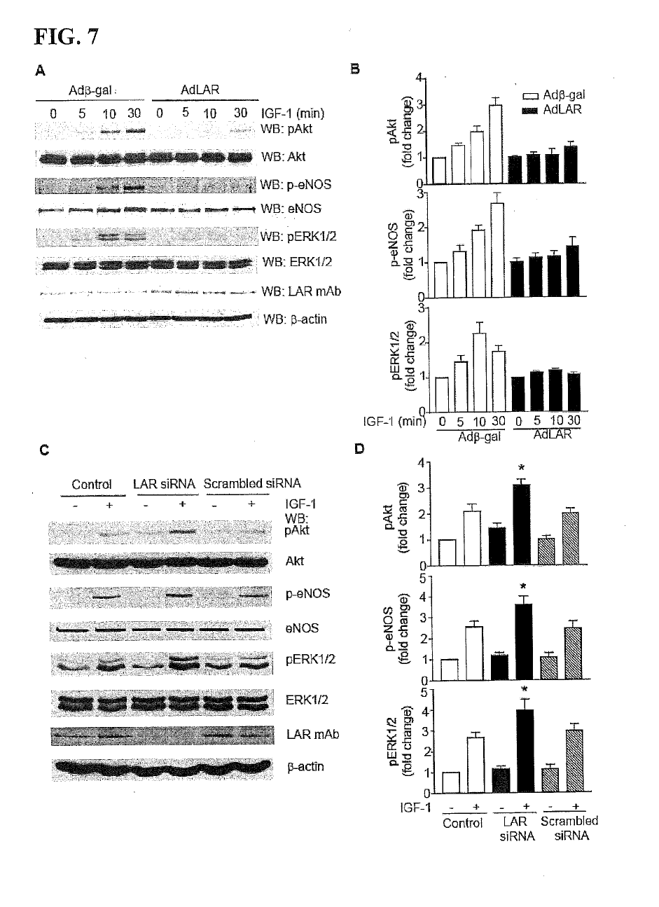

Phosphatidylinositol-3-kinase (PI3K)/Akt/eNOS and ERK1/2/eNOS/NO pathways play a pivotal role in regulating angiogenesis and arteriogenesis (Rikitake et al., Arterioscler. Thromb. Vasc. Biol. 22:108 (2002); Urano et al., Arterioscler. Thromb. Vasc. Biol. 28:827 (2008)). Because these signaling pathways are induced by many angiogenic growth factors, the effect of LAR overexpression on the activation of these pathways was examined in HUVECs treated with IGF-1 and VEGF. IGF-1 treatment increased the phosphorylation of Akt, eNOS and ERK1/2 in a time-dependent manner in Adβ-gal infected HUVECs, and this increase was abrogated in cells infected with 2.5 MOI of AdLAR (FIGS. 7A and 7B). In contrast, LAR siRNA significantly enhanced IGF-1-induced phosphorylation of Akt, eNOS and ERK1/2 in HUVECs compared with control or scrambled siRNA infected cells (P<0.01; FIGS. 7C and 7D). LAR overexpression (FIG. 2) or LAR siRNA (FIG. 3) had no effect on the phosphorylation of Akt, eNOS and ERK1/2 induced by VEGF in HUVECs. These data support the argument that LAR is a specific negative regulator of IGF-1Rβ autophosphorylation as LAR expression levels modulated IGF-1Rβ downstream signaling pathways.

### Example 5

### LAR Negatively Regulates IGF-1-Stimulated EC Proliferation and Migration

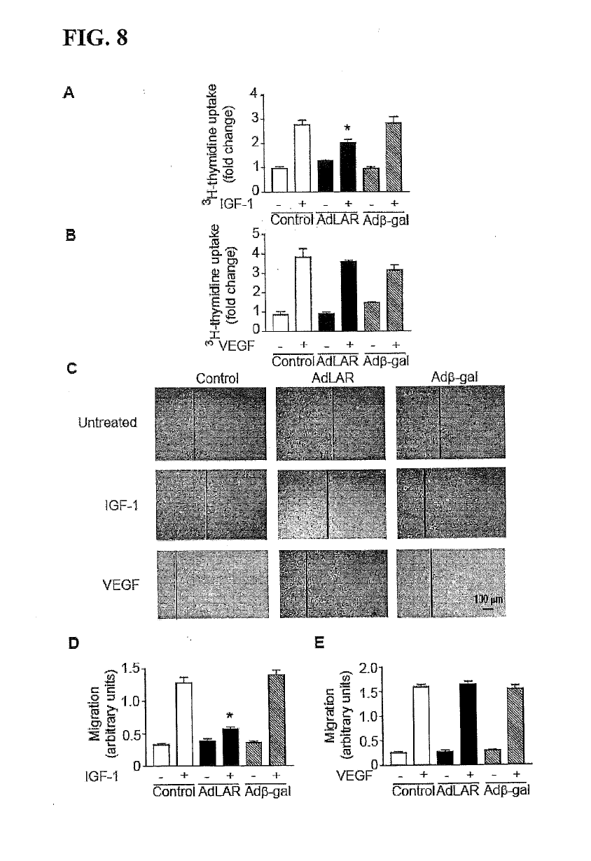

Because in vitro and in vivo studies indicate that IGF-1 induces EC proliferation and migration (Grant et al., Ann. NY Acad. Sci. 692:230 (1993)), and the data above show that LAR levels modulate IGF-1-induced activation of ERK1/2 which coordinate proliferation and migration of ECs during angiogenesis, the effects of overexpression of LAR on IGF-1-induced EC proliferation and migration was examined. Uninfected (control) and AdLAR or Adβ-gal infected HUVECs were either left untreated or treated with IGF-1 and DNA synthesis was assayed by measuring [³H]-thymidine uptake. IGF-1-induced DNA synthesis was decreased by approximately 30% in LAR overexpressing cells compared with uninfected or Adβ-gal infected HUVEC (P<0.05; FIG. 8A). Neither AdLAR nor Adβ-gal infection affected basal cell proliferation. In contrast to its effect on IGF-1-induced proliferation, LAR overexpression had no effect on VEGF-stimulated proliferation of HUVECs (FIG. 8B).

Uninfected and AdLAR or Adβ-gal infected HUVECs were used in a scratch wound assay to assess the effect of LAR overexpression on IGF-1-stimulated migration of HUVECs. Migration was analyzed 16 h after scratching because proliferation was not a contributing factor for wound healing at this time. Compared with uninfected or Adβ-gal infected cells, IGF-1 stimulated migration was significantly attenuated in AdLAR infected HUVECs (P<0.001: (FIGS. 8C and 8D). Neither AdLAR nor Adβ-gal infection affected basal cell migration. In contrast, LAR overexpression had no effect on VEGF-induced migration of HUVECs (FIGS. 8C and 8E). These data further support the specific, negative regulatory effects of LAR on IGF-1-induced EC function.

### Example 6

### LAR Modulates IGF-1 Stimulated Angiogenesis In Vitro

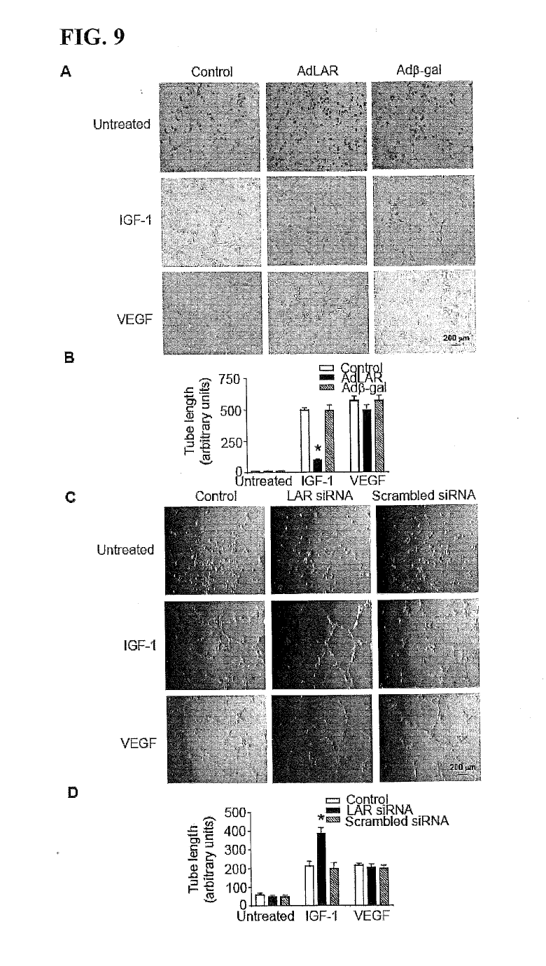

It was examined whether LAR expression levels in HUVECs alter tube-forming activity, an in vitro indicator of angiogenic potential. To address this, control, Adβ-gal or AdLAR infected HUVECs were seeded on Matrigel and treated with or without IGF-1. Control and Adβ-gal infected HUVECs formed an organized network of endothelial tubes within 5 h of treatment with IGF-1. In contrast, LAR overexpression significantly inhibited IGF-1-induced tube formation (P<0.001 vs IGF-1 treated control or Adβ-gal-infected HUVECs (FIGS. 9A and 9B). Capillary nodes in LAR overexpressing HUVECs were surrounded by clusters of ECs that failed to assemble into tubes. But, VEGF-induced tube formation in LAR overexpressing HUVECs remained unchanged compared with that in control and Adβ-gal infected HUVECs (FIGS. 9A and 9B). Consistent with IGF-1R-specific negative regulatory effect of LAR on endothelial function, knockdown of LAR using siRNA increased IGF-1-induced tube formation by 81% compared with that in control and Adβ-gal infected HUVECs (P<0.001; FIGS. 9C and 9D). As predicted, LAR siRNA had no effect on VEGF-induced tube formation (FIGS. 9C and 9D).

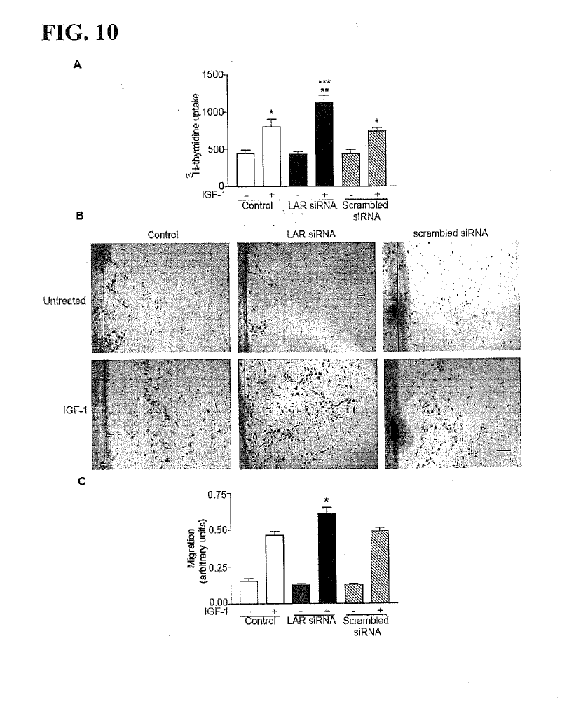

Additional evidence for the negative regulatory effect of LAR on IGF-1 induced EC proliferation was obtained by LAR siRNA transfection studies. Control HUVECs and LAR siRNA or scrambled siRNA transfected HUVECs were either left untreated or treated with IGF-1, and DNA synthesis was assayed by measuring [³H]-thymidine uptake. IGF-1 induced DNA synthesis was increased by approximately 39% in LAR siRNA transfected cells compared with scrambled siRNA transfected cells (P<0.01; FIG. 10A). Similarly, IGF-1 stimulated cell migration was significantly enhanced in LAR siRNA transfected HUVECs compared with untransfected or scrambled siRNA transfected cells, in scratch wound assays (P<0.05; FIGS. 10B and 10C). These results complement the data obtained with LAR overexpression studies, confirming the modulatory effect of LAR protein levels on IGF-1 induced EC function.

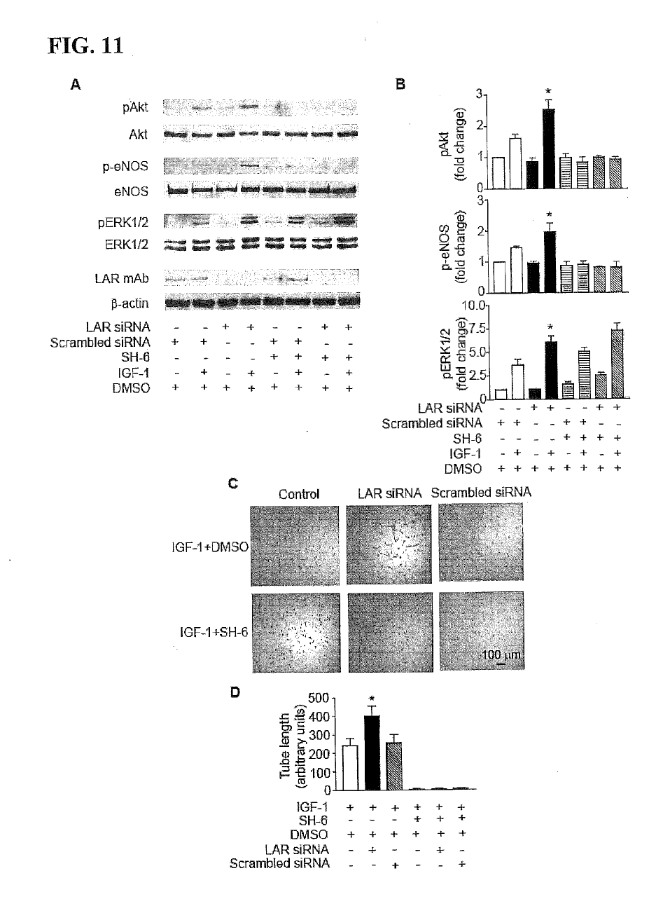

To test the relative roles of IGF-1Rβ downstream signaling pathways—PI3K/Akt/eNOS and PI3K/ERK1/2—in LAR regulated angiogenesis, it was first determined whether these pathways are sequentially regulated in HUVECs treated with IGF-1. Scrambled or LAR siRNA infected HUVECs were pretreated with 1 μmol/L, SH-6, an Akt inhibitor and then treated with or without IGF-1. SH-6 pretreatment abrogated enhanced activity of Akt and eNOS in LAR siRNA infected HUVECs treated with IGF-1, but had no effect on ERK1/2 (FIGS. 11A and 11B). SH-6 also abrogated enhanced IGF-1-induced endothelial tube formation in LAR siRNA infected HUVECs (FIGS. 11C and 11D), which indicates that PI3K/Akt/eNOS pathway plays a predominant role in angiogenesis.

### Example 7

### LAR Modulates IGF-1 Stimulated Angiogenesis Ex Vivo and In Vivo

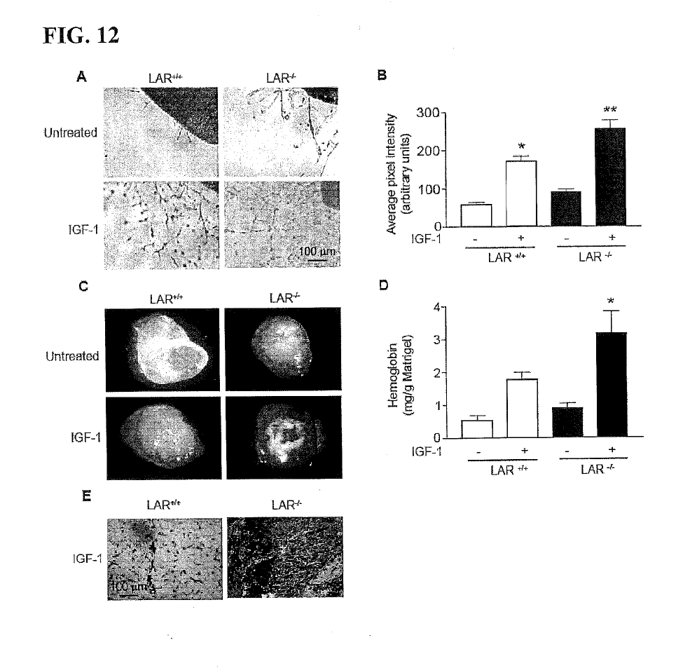

To test the functional relevance of LAR-regulated IGF-1-induced endothelial tube formation, ex vivo vascular sprouting was examined using aortic rings from wild-type and LAR⁻⁻ mice. Sections of mouse aorta were cultured in growth factor-reduced Matrigel and the length of vascular sprouts was measured on day 10. Mean length of newly sprouted vessels in LAR^{-/-} mice was increased by about 55% compared with that in wild-type mice. IGF-1 treatment significantly enhanced the mean length of newly sprouted vessels from the aortic rings from both wild-type (P<0.05 vs respective control) and LAR^{-/-} mice (P<0.01 vs respective control) (FIGS. 12A and 12 B). However, IGF-1-induced increase in the mean length of sprouted vessels from the aortic rings of LAR^{-/-} mice was significantly higher than that from the wild-type mice (P<0.001). These data together with EC tube formation results suggest that LAR regulates IGF-1-responsive angiogenesis in vivo.

Next, it was examined whether the absence of LAR results in enhanced IGF-1-induced angiogenesis in vivo using the Matrigel plug assay. Five hundred μl of Matrigel supplemented with IGF-1, basic fibroblast growth factor and heparin or basic fibroblast growth factor and heparin alone were injected subcutaneously into wild-type and LAR^{-/-} mice. Matrigel plugs recovered after 10 days from IGF-1 treated LAR^{-/-} showed significantly increased hemoglobin content compared to plugs from wild-type mice (P<0.05; FIGS. 12C and 12D). Linear structures formed with cells whose lumens are filled with red blood cells were more abundant in sections of plugs from LAR^{-/-} mice compared to wild-type mice (FIG. 12E). These data indicate that these structures are the outgrowth of the vasculature and support the notion that LAR regulates IGF-1-induced angiogenesis in vivo.

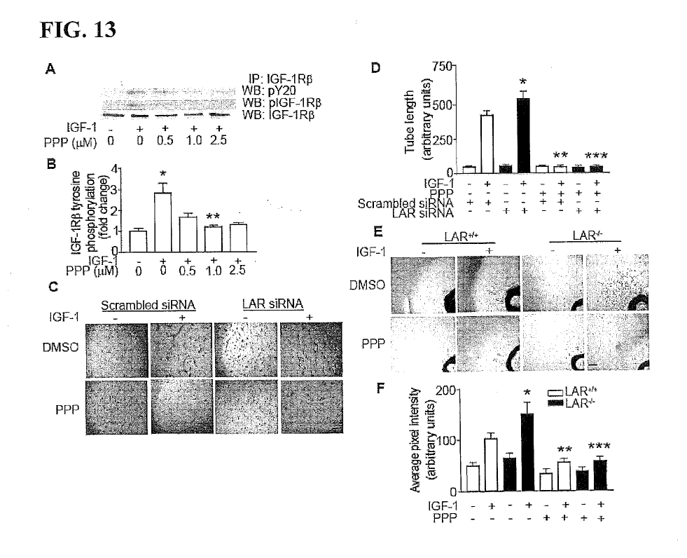

To demonstrate that enhancement of IGF-1-induced angiogenesis in the aortic ring and Matrigel plug assays of LAR^{-/-} mice is specifically due to increase in IGF-1R signaling, the effect of picropodophyllin (PPP), a selective IGF-1Rβ kinase inhibitor, on IGF-1 induced phosphorylation of IGF-1Rβ was first tested. Immunoprecipitation/Western analysis using anti-IGF-1Rβ/pY20 antibodies and Western analysis using antibody against phospho-IGF-1Rβ (pYpY1135/1136) showed a dose-dependent inhibition of IGF-1Rβ autophosphorylation in HUVECs treated with IGF-1, with significant inhibition observed at 1 μM PPP (FIGS. 13A and 13B). If enhanced IGF-1-induced angiogenesis in LAR^{-/-} mice is specifically due to increase in IGF-1Rβ signaling, then inhibition of IGF-1Rβ autophosphorylation with PPP should inhibit cord-forming activity of HUVECs in vitro. To address this, scrambled siRNA or LAR siRNA transfected HUVECs wee seeded on Matrigel and treated with or without IGF-1 in the presence and absence of 1 μM PPP (FIGS. 13C and 13D). PPP abrogated IGF-1-induced cord formation in both scrambled siRNA and LAR siRNA transfected HUVECs. Similarly, 1 μM PPP significantly inhibited the mean length of sprouts from IGF-1 treated wild-type (P<0.05) and LAR^{-/-} (P<0.001) aortic rings cultured in Matrigel medium (FIGS. 13E and 13F).

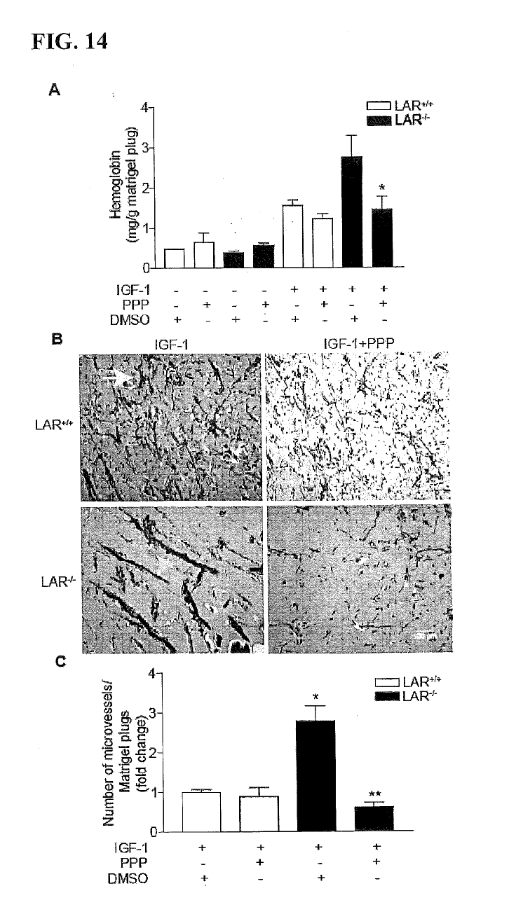

Next, the effect of PPP on IGF-1-induced in vivo angiogenesis was examined using the Matrigel plug assay as described above. Although the hemoglobin content of IGF-1 embedded Matrigel plugs decreased in both wild-type and LAR^{-/-} mice in the presence of PPP, the decrease was statistically significant in LAR^{-/-} mice (P=0.01; FIG. 14A). In addition, the number of microvessels in IGF-1 embedded Matrigel plugs in LAR^{-/-} mice was significantly decreased (P<0.001; FIGS. 14B and 14C) and the vessels were devoid of red blood cells. These data further support the notion that LAR modulates IGF-1-induced angiogenesis by regulating IGF-1Rβ signaling.

### Example 8

### Blood Flow Recovery After Femoral Artery Ligation is Faster in the Absence of LAR

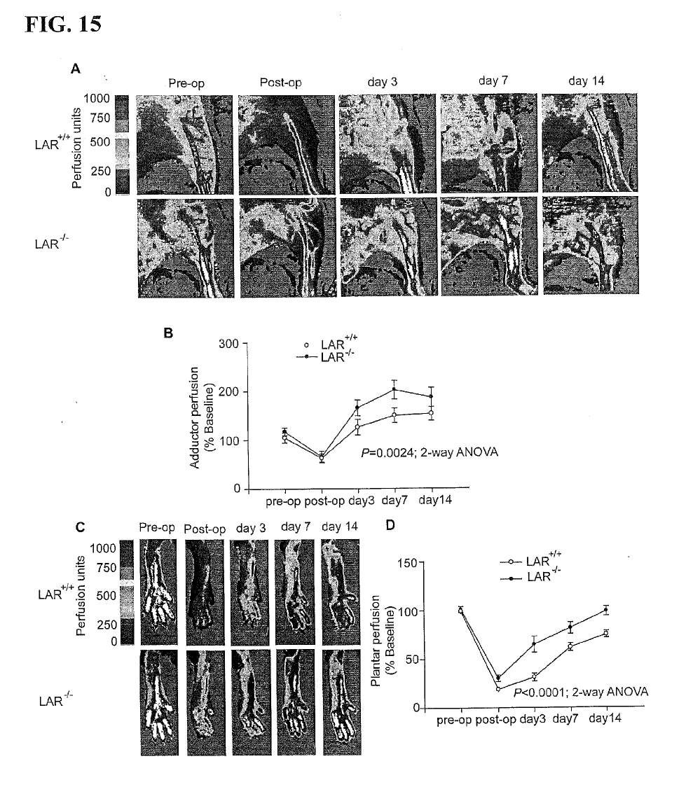

To determine whether LAR deficiency affects angiogenesis in vivo under pathophysiological conditions, as suggested by the above findings, blood flow in the hindlimb was examined after femoral artery ligation. Recovery of perfusion was significantly better in the adductor muscle of LAR^{-/-} mice compared with wild-type mice after femoral artery ligation (P=0.0024, 2-way ANOVA; (FIGS. 15A and 15B). Plantar perfusion, which correlates with overall collateral-dependent flow (Chalothorn et al., Am. J. Physiol. Heart Circ. Physiol. 289:H947 (2005)), was also significantly greater in LAR^{-/-} compared to the wild-type mice (P<0.001, 2-way ANOVA; FIGS. 15C and 15D). Because IGF-1 expression is increased after femoral artery ligation (Paoni et al., Physiol. Genomics 11:263 (2002)), the milder ischemia observed in LAR^{-/-} mice compared to the wild-type mice is consistent with increased IGF-1-induced angiogenesis observed in vitro and ex vivo in the absence of LAR.

### Example 9

### LAR Regulates Capillary Density, Number of Preexisting Collaterals and Collateral Remodeling after Femoral Artery Ligation

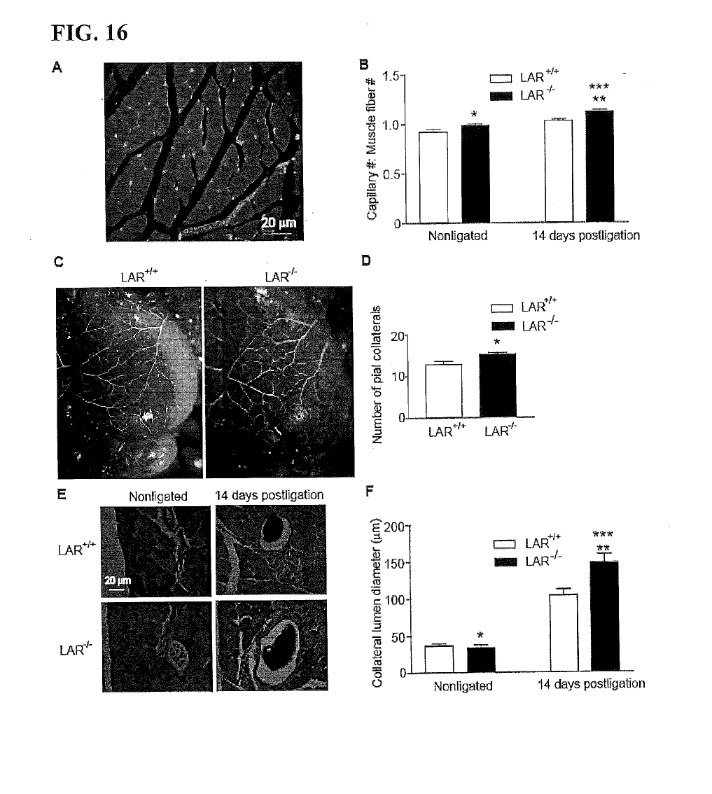

Changes in plantar perfusion partly reflect anatomic differences in capillary density and preexisting collateral density because vascular tone is strongly inhibited in the ischemic limb (Yang et al., Am. J. Physiol. Heart Circ. Physiol. 282:H301 (2002)). To determine whether LAR deficiency had an effect on capillary density, capillary-to-muscle fiber ratio in the gastrocnemius muscle was determined before and 14 days after femoral artery ligation. A significant increase in capillary density was observed in LAR^{-/-} mice compared with wild-type mice under basal conditions (P<0.001; FIGS. 16A and 16B). After ligation, capillary density remained unchanged in wild-type mice. But, capillary density increased significantly after ligation in LAR^{-/-} mice (P<0.01).

Plantar flow in LAR^{-/-} dropped less immediately after femoral artery ligation (FIG. 16D), suggesting more abundant collaterals, compared to wild-type. To determine whether LAR deficiency had an effect on collateral density, X-ray angiography was performed immediately and 14 days after ligation to detect native collaterals and smaller collaterals formed after the onset of remodeling, respectively. Vessels crossing a line drawn through the middle of the adductor collateral zone were counted following Rentrop criteria as described previously (Clayton et al., Circ. Res. 103:1027 (2008)). No difference was observed between the wild-type and LAR^{-/-} mice in either the native collateral density or collateral number post ligation. Because angiography is limited in resolution, we also determined collateral number in the cerebral pial circulation in wild-type and LAR^{-/-} mice. Unlike in hindlimb, all collaterals in the cerebral cortical circulation are confined to the pial surface and thus can be quantified with high fidelity (Clayton et al., Circ. Res. 103:1027 (2008)). Also, collateral density in this tissue qualitatively predicts collateral density in other tissues in the mouse (Clayton et al., Circ. Res. 103:1027 (2008)). Adult LAR^{-/-} mice possess more collaterals than wild-type mice (P<0.05; FIGS. 16C and 16D). These results suggest that LAR negatively regulates both basal capillary and collateral density.

Because formation of large collateral conductance arteries, by the remodeling of preexistent arteriolar collateral networks (“arteriogenesis”), is the most efficient mechanism for restoring blood flow after arterial occlusion (Hershey et al., Cardiovasc. Res. 49:618 (2001)), collateral diameter was measured before and 14 days after ligation. Basal collateral lumen diameters were significantly smaller in LAR^{-/-} mice compared with wild-type mice (P<0.001; FIGS. 16E and 16F). Collateral lumen diameters were increased 2.9-fold in wild-type mice 14 days after femoral artery ligation, but the increase was not statistically significant. But, the absence of LAR significantly increased collateral lumen diameters following femoral artery ligation (P<0.001). Collateral lumen diameters were significantly larger in LAR^{-/-} mice compared with wild-type mice 14 days after femoral artery ligation. These data suggest that LAR regulates collateral remodeling under ischemic conditions.

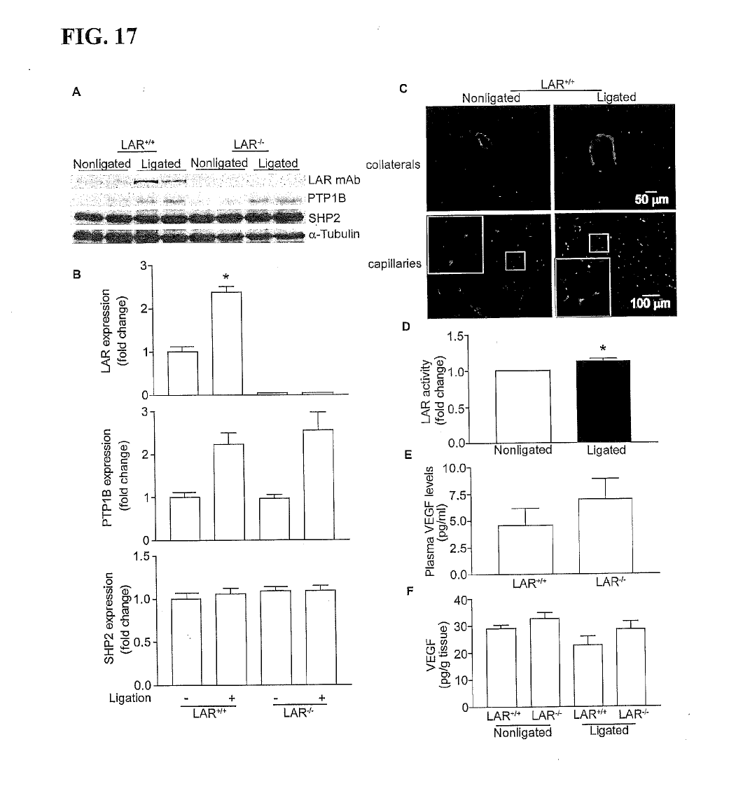

To assess the role of LAR on ischemic neovascularization in vivo, the expression and activity of LAR was examined in the hindlimb after femoral artery ligation. Western blot analysis shows that expression of LAR protein was robustly increased in the ischemic hindlimb of wild-type mice at 7 days after ligation compared with that in the non-ligated hindlimbs (P<0.001; FIGS. 17A and 17B). Interestingly, PTP1B expression was increased similarly in wild-type and LAR^{-/-} mice, whereas SHP2 protein levels were unchanged in the hindlimbs of both wild-type and LAR^{-/-} mice after ligation. Increase in LAR expression was also evident around remodeled collaterals in the gracilis muscle and capillaries in gastrocnemius of wild-type mice at 21 days after ligation (FIG. 17C). In addition, a significant increase in LAR activity was observed in the hindlimb of wild-type mice at 7 days after femoral artery ligation (FIG. 17D). Interestingly, plasma VEGF expression trended higher (P=0.0578) in LAR^{-/-} mice compared to wild-type mice 7 days after femoral artery ligation (FIG. 17E). There was no significant difference in skeletal muscle VEGF levels before and after femoral artery ligation in both wild-type and LAR^{-/-} mice (FIG. 17F).

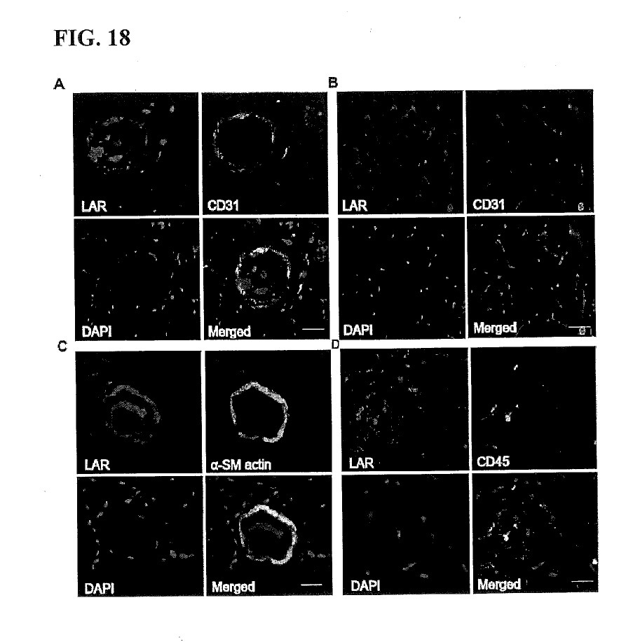

To further assess the role of LAR in neovascularization, LAR expression was examined in endothelial cells (FIGS. 18A and 18B), smooth muscle cells (FIG. 18C) and macrophages (FIG. 18D) of wild-type mice 21 days after femoral ligation. Immunofluorescence labeling of adductor (FIG. 18A) and gastrocnemius (FIG. 18B) sections with CD31 (green) and LAR (red) antibodies showed colocalization (yellow) of LAR with CD31-positive cells, which indicates robust LAR expression in endothelial cells of collaterals and capillaries 21 days after the femoral artery ligation. Similarly, immunofluorescence staining of muscle tissues with α-SM actin (green) and LAR (red) antibodies showed colocalization (yellow) of these two proteins (FIG. 18C) which suggests that smooth muscle cells of collaterals express LAR 21 days after the femoral artery ligation. In contrast, there were very few CD45-positive cells 7 and 21 days after the femoral artery ligation. CD45-positive cells, not observed in the muscle tissue of the contralateral hindlimb, were present in the ischemic muscle tissue at day 3 after the femoral artery ligation and the CD45 immunostaining was colocalized with LAR immunostaining in the collaterals (FIG. 18D). These data suggest mobilization of macrophages and other bone marrow-derived cells in response to ischemia, though no difference in the recruitment of these cells to the perivascular areas of muscle tissue was detected between wild-type and LAR^{-/-} mice. Collectively, these data indicate that LAR expression and activity are significantly increased in the ischemic muscle tissue, LAR is robustly expressed in endothelial cells of collaterals and capillaries and smooth muscle cells and macrophages of collaterals of wild-type mice and LAR is a negative regulator of neovascularization under ischemic conditions.

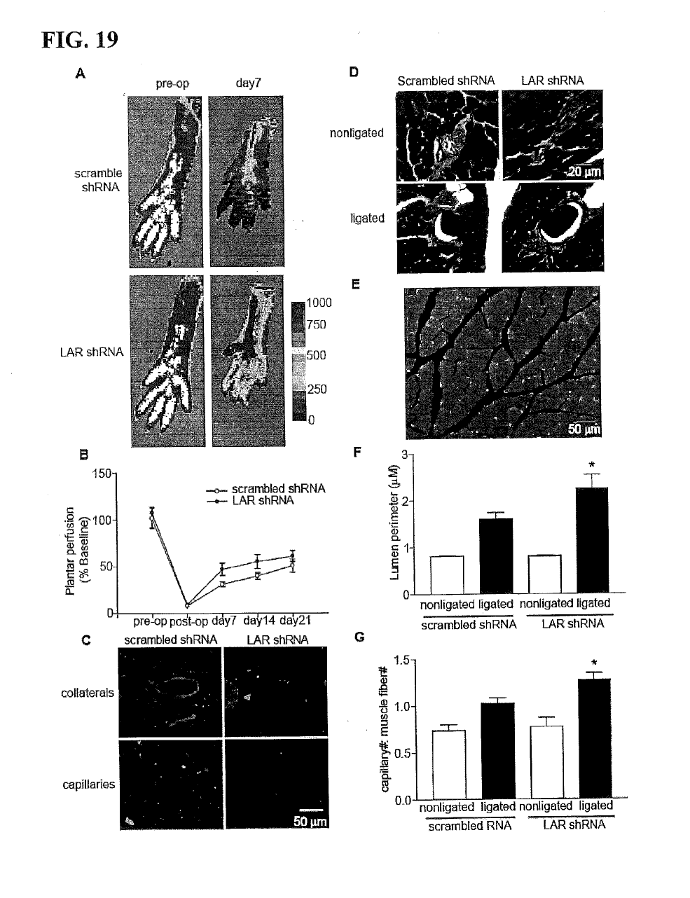

To determine whether suppression of LAR expression improves recovery of perfusion after arterial obstruction, blood flow was examined in the ischemic hindlimbs of db/db mice injected with lentiviral scrambled shRNA or lentiviral LAR shRNA. For lentiviral transduction studies, 1×10⁹ plaque-forming units of lentivirus were injected into several sites in the adductor and the gastrocnemius muscle. Lentiviral LAR shRNA transduction significantly suppressed LAR expression in the collaterals and capillaries of ischemic hindlimb as shown by immunohistochemistry (FIG. 19C), Western analysis, and quantitative real-time reverse-transcription PCR analysis on day 21 after femoral artery ligation. Laser Doppler perfusion analysis showed that recovery of plantar perfusion was significantly improved in mice treated with lentiviral LAR shRNA compared with scrambled shRNA (P=0.0155; two-way ANOVA; FIGS. 19A and 19B). Consistent with this, suppression of LAR expression significantly augmented collateral remodeling (P=0.05; FIGS. 19D and 19F) and capillary density (FIG. 19E) (P=0.05; FIG. 19G) 21 days after femoral ligation in db/db mice transduced with lentiviral LAR shRNA. Together, these data support the notion that suppression of LAR expression improves postischemic recovery of perfusion in db/db mice by enhancing angiogenesis and arteriogenesis.

### Example 10

### Quantitation of Endothelial Progenitor Cells

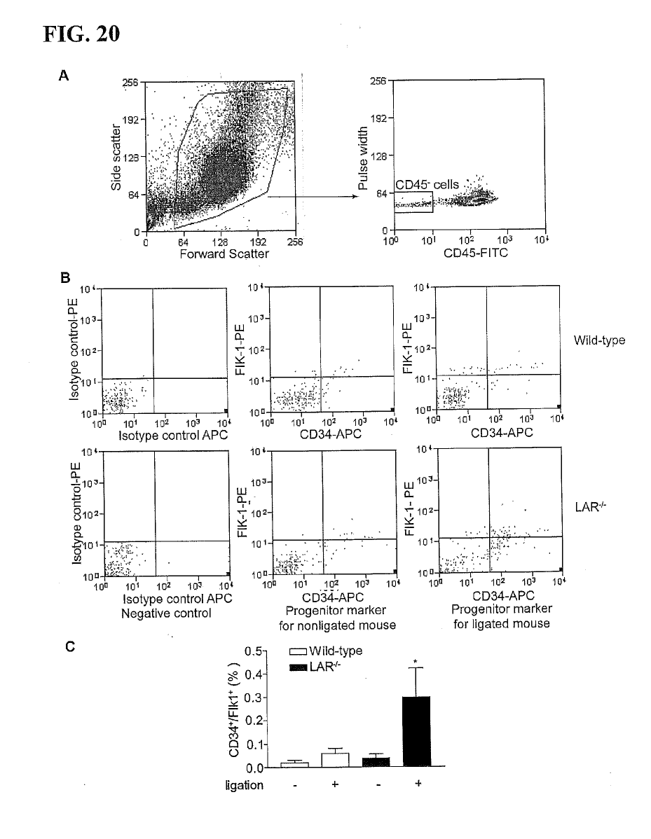

CD34⁺/Flk-1⁺ endothelial progenitor cells (EPCs) play a critical role in neovascularization of ischemic tissue (Kalka et al., Proc. Natl. Acad. Sci, USA 97:3422 (2000); Kuiszewski et al., Mol. Ther. 19:895 (2011). To determine whether enhanced neovascularization in LAR^{-/-} mice following femoral artery ligation is associated with increased mobilization of EPCs, mononuclear cells were isolated from peripheral blood cells and then analyzed for the CD34⁺ and Flk-1⁺ population. FACS (Fluorescence-activated cell sorting) analysis showed that a vast majority of the mononuclear cells are CD45⁺ (>99%) whereas only a small proportion are CD34⁺ (0.3%) (FIG. 20A). In nonligated mice, CD34⁺/Flk-1⁺ cells comprised only 0.02±0.01% of the cells in the peripheral blood (FIG. 20A). After unilateral ligation of the common femoral artery, the number of CD34⁺/Flk-1⁺ cells in peripheral blood from LAR^{-/-} mice significantly increased as compared with the non-ligated control (*P<0.05), whereas the increase in the number of CD34⁺/Flk-1⁺ cells in the ligated wild-type mice on day 3 was not significant as compared with the nonligated mice (FIG. 20B). These data indicate that enhanced EPC mobilization might contribute to enhanced neovascularization after femoral artery ligation in LAR^{-/-} mice.

Despite the importance of IGF-1R stimulated signaling pathways in multiple fundamental cellular processes, not much is known about the mechanisms that regulate the activity of this RTK. The studies above demonstrate that 1) LAR binds to IGF-1Rβ but not VEGFR2 in HUVECs under basal conditions and the affinity of LAR to IGF-1Rβ is enhanced by the autophosphorylation of the receptor; 2) overexpression of LAR inhibits IGF-1-induced IGF-1Rβ autophosphorylation, activation of downstream signaling proteins Akt, eNOS and ERK1/2, and proliferation and migration of ECs, whereas knockdown of LAR using siRNA significantly enhances these responses; 3) LAR expression levels regulate IGF-1-induced angiogenesis in vitro, ex vivo and in vivo; 4) blood flow recovery is increased in LAR^{-/-} mice compared to the wild-type mice under ischemic conditions; and 5) LAR expression regulates capillary density, preexisting collateral number and collateral remodeling after femoral artery ligation.

It has been reported that IGF-1R activity is modulated by SHP2 PTP in VSMCs (Maile et al., J. Biol. Chem. 277:8955 (2002)), but muscle specific knockdown of SHP2 resulted in insulin resistance which was attributed to impaired activities of downstream signaling components PKC-ζ/λ, and AMP-activated protein kinase (Princen et al., Mol. Cell. Biol. 29:378 (2009)). The data from pulldown and coprecipitation assays as well as overexpression and knockdown experiments in vitro and ex vivo demonstrate that LAR is a negative regulator of IGF-1R autophosphorylation in ECs and support earlier findings regarding LAR and IGF-1R interaction (Niu et al., J. Biol. Chem. 282:19808 (2007)). Although LAR binds to several cellular substrates (Kulas et al., J. Biol. Chem. 271:748 (1996)), the data above indicating that LAR does not inhibit PDGFR and VEGFR activities are in agreement with the growing body of evidence that PTP-RTK interactions are specific (Niu et al., J. Biol. Chem. 282:19808 (2007); Ostman et al., Trends Cell Biol. 11:258 (2001)).

Down regulation of LAR significantly altered tube formation and vascular sprouting in two separate angiogenesis assays. The mean length of tubes in response to IGF-1 treatment was significantly higher in the absence of LAR. Of note, the time for tube formation was only 5 h, which indicates that cellular proliferation is not involved in this morphogenesis. LAR overexpression markedly attenuated tube formation, an effect that could not be attributed to diminished EC survival as LAR expression levels did not affect cell survival. However, enhanced EC proliferation and migration may have contributed to the enhanced in vivo angiogenesis observed in response to IGF-1 treatment in the absence of LAR.

It is possible that other proteins contributed to the increased angiogenesis in LAR knockout mice under ischemic conditions because of the potential for changes in the expression of an array of proteins in the global knockout models. However, the in vivo Matrigel data showing increased IGF-1-induced angiogenesis in LAR knockout mice and in vitro data indicating enhanced angiogenic signaling pathways and in vitro and ex vivo angiogenesis in the absence of LAR firmly establish a role for this PTP in modulating angiogenesis.

Decreased arteriogenesis was observed in the ischemic brains of eNOS-deficient mice (Cui et al., Neuroscience 159:744 (2009)) and inhibition of eNOS reduces ischemic coronary collateral growth (Matsunaga et al., Circulation 102:3098 (2000)). The data above are consistent with these results as LAR deficiency, which stimulates PI3K/Akt/eNOS pathway, resulted in not only increased capillary density but also increased native (baseline) pial collaterals. The failure to detect this difference in hindlimb with X-ray angiography may reflect the lower resolution of the latter procedure (Clayton et al., Circ. Res. 103:1027 (2008)). However, it is supported by the smaller drop in hindlimb perfusion immediately after femoral artery ligation. The significant increase observed in capillary-to-fiber ratio at day 14 after femoral artery ligation could contribute to the enhanced recovery of hindlimb perfusion in the LAR-deficient mice, although native pre-existing collateral number and subsequent collateral remodeling are considered the primary determinants of perfusion recovery (Clayton et al., Circ. Res. 103:1027 (2008)). Medial SMCs are the vital participants in collateral remodeling (Cai et al., Mol. Cell. Biochem. 264:201 (2004)) and the switch from the contractile to the synthetic phenotype is integral to this process. It is noteworthy that we have reported increased VSMC proliferation in the absence of LAR (Niu et al., J. Biol. Chem. 282:19808 (2007)).

In conclusion, this is the first demonstration that LAR negatively regulates IGF-1R signaling in endothelial cells involved in angiogenesis and that deficiency of LAR enhances vasculogenesis and arteriogenesis under ischemic conditions. Because PTP activity is increased in peripheral ischemic diseases, these data indicate that localized inhibition of LAR may have therapeutic benefit.

The foregoing is illustrative of the present invention, and is not to be construed as limiting thereof. The invention is defined by the following claims, with equivalents of the claims to be included therein.

1-2. (canceled)

3. A method of increasing angiogenesis and/or arteriogenesis in a tissue of a subject, comprising decreasing the expression and/or activity of LAR in said tissue of said subject.

4. A method of treating or preventing ischemia in a tissue of a subject, comprising decreasing the expression and/or activity of LAR in said tissue of said subject.

5. The method of claim 3, wherein said subject has diabetes.

6. The method of claim 3, wherein said subject has cardiovascular or cerebrovascular disease or has experienced ischemia or stroke.

7. The method of claim 3, wherein said subject is at risk for ischemia.

8. The method of claim 3, wherein said subject has a graft or other transplanted tissue, anastomosis, wound, ulcer, burn, male pattern baldness, atherosclerosis, ischemic heart tissue, ischemic peripheral tissue, myocardial or cerebral infarction, or vascular occlusion or stenosis.

9. The method of claim 3, comprising delivering an inhibitor of LAR expression and/or activity to said tissue.

10. The method of claim 9, wherein said tissue is in a limb.

11. The method of claim 10, wherein said tissue is in a foot or toe.

12. The method of claim 9, wherein said tissue is in the heart or brain.

13. The method of claim 3, wherein decreasing the expression and/or activity of LAR comprises decreasing the level of a nucleic acid encoding LAR.

14. The method of claim 13, wherein the method comprises delivering an antisense RNA to said subject to decrease the level of a nucleic acid encoding LAR.

15. The method of claim 13, wherein the method comprises delivering an siRNA to said subject to decrease the level of a nucleic acid encoding LAR.

16. The method of claim 3, wherein decreasing the expression and/or activity of LAR comprises decreasing the activity of LAR polypeptide.

17. The method of claim 16, wherein the method comprises delivering an antibody that specifically binds LAR to said subject to decrease the activity of LAR.

18. The method of claim 17, wherein said antibody is a monoclonal antibody.

19. The method of claim 16, wherein the method comprises delivering an aptamer that specifically binds LAR to said subject to decrease the activity of LAR.

20. The method of claim 16, wherein the method comprises delivering a small molecule that inhibits LAR to said subject to decrease the activity of LAR.

21. A method of identifying a compound that regulates angiogenesis and/or arteriogenesis, comprising determining the expression and/or activity of LAR in the presence and absence of a test compound, and selecting a compound that increases or decreases the level of expression and/or activity of LAR relative to the level in the absence of the compound, thereby identifying the compound as a compound that regulates angiogenesis.

22. The method of claim 21, wherein at least part of the method is carried out in a cell.

23. The method of claim 22, wherein said cell is an endothelial cell.


SEQUENCE LISTING (4 sequences)

SEQ ID NO: 1 - RNA, 19 nt, Artificial
  siRNA sequence
ccacuaugcaacguaacua

SEQ ID NO: 2 - RNA, 19 nt, Artificial
  siRNA sequence
cagacgagauccuguuuca

SEQ ID NO: 3 - RNA, 19 nt, Artificial
  siRNA sequence
ggcuaagucaagaucaaca

SEQ ID NO: 4 - DNA, 57 bp, Artificial
  shRNA Sequence
ccggcgctttgaggtaattgagtttctcgagaaactcaattacctcaaagcgttttt